(19) United States
(12) Patent Application Publication
(10) Pub. No.: US 2026/0265300 A1
(43) Pub. Date: Sep. 10, 2026

(54) TARGETING PRAC1 IN STEROID HORMONE DRIVEN CANCER

(71) Applicants: Fred Hutchinson Cancer Center, Seattle, WA (US); Board of Regents, The University of Texas System, Austin, TX (US)

(72) Inventors: Michael Haffner, Seattle, WA (US); Jin-Yih Low, Seattle, WA (US); Elizabeth Wasmuth, Seattle, WA (US); Chitvan Mittal, Seattle, WA (US)

(73) Assignees: Fred Hutchinson Cancer Center, Seattle, WA (US); Board of Regents, The University of Texas System, Austin, TX (US)

(21) Appl. No.: 19/166,806

(22) PCT Filed: Mar. 21, 2024
(86) PCT No.: PCT/US2024/021012

§ 371 Date: Sep. 18, 2025

Related U.S. Application Data
(60) Provisional application No. 63/491,458, filed on Mar. 21, 2023.

(51) Int. Cl. C07K 7/06; A61K 38/00; A61K 45/06; A61P 35/00; C12N 15/86; C12Q 1/6809; C12Q 1/6886

(52) U.S. Cl. CPC C07K 7/06; A61K 45/06; A61P 35/00; C12N 15/86; C12Q 1/6809; C12Q 1/6886; A61K 38/00; C12N 2740/10041; C12N 2740/15043; C12Q 2600/106

(57) ABSTRACT

In some embodiments, the present disclosure provides steroid hormone receptor ligand binding peptides comprising at least 6 contiguous amino acid residues found within the carboxy terminal receptor binding domain of the PRAC1 protein. Methods for the use of the peptides and their derivatives, pharmaceutical compositions comprising the peptides and their derivatives in treating a steroid hormone driven cancer and inhibiting the interaction between the steroid hormone receptor, its co-factors, and its ligand are also provided. Additionally, provided herein is a method of overcoming resistance or restoring sensitivity to a therapy targeting the androgen receptor signaling axis in a subject suffering from a steroid hormone driven cancer.

### CROSS-REFERENCE TO RELATED APPLICATION

This application claims the benefit of U.S. Provisional Application No. 63/491,458, filed Mar. 21, 2023, the disclosure of which is incorporated herein by reference in its entirety.

### STATEMENT OF GOVERNMENT LICENSE RIGHTS

This invention was made with Government support under W81XWH-22-1-0199 awarded by the United States Army Medical Research and Development Command. The government has certain rights in the invention.

### STATEMENT REGARDING SEQUENCE LISTING

The Sequence Listing XML associated with this application is provided in XML format and is hereby incorporated by reference into the specification. The name of the XML file containing the sequence listing is 1896-P86WO_Seq_List_20240321.xml. The XML file is 110,687 bytes; was created on Mar. 21, 2024; and is being submitted electronically via Patent Center with the filing of the specification.

### BACKGROUND

Steroid hormone receptors are a group of nuclear receptors that share structural and functional similarities and contribute to key physiological functions. The estrogen and androgen receptors are crucially involved in the development of the male and female reproductive systems and ensure reproductive activity post-puberty. In addition to their important role in male and female physiology, steroid receptors (particularly the androgen, estrogen, and progesterone receptors) have been shown to be important contributors to tumor progression and growth in steroid hormone-driven malignancies. For instance, in breast, uterine carcinomas and certain types of ovarian tumors, the estrogen receptor exerts a context-dependent pro-proliferative function. This insight has led to specific treatment strategies that target estrogen receptor function. Indeed, drugs that target the estrogen receptor or decrease estrogen hormone production are the mainstay of therapy for most breast, and uterine cancers.

Similarly, the growth-promoting effect of estrogen in cancers arising from female reproductive organs, prostate cancer (PC), is an androgen-dependent tumor, and the androgen receptor (AR) drives numerous mechanisms involved in disease progression. The standard therapy for metastatic PC involves various pharmacological approaches that inhibit the AR signaling axis. Despite initially profound responses to androgen deprivation therapies, most patients progress to castration-resistant prostate cancer (CRPC). Highly potent second-generation AR signaling inhibitors (ARSIs), such as abiraterone acetate and enzalutamide, that were developed to overcome this resistance have shown improvements in outcomes, but essentially all patients develop resistance to these agents. Much of the mortality associated with advanced PC can be attributed to resistance to AR signaling inhibitors (ARSIs). Therefore, to improve therapy outcomes, there is a great need to better understand mechanisms involved in treatment resistance and to develop novel strategies to counteract their emergence.

Broadly, considering the proven clinical efficacy of treating steroid hormone-driven cancers with agents targeting the estrogen receptor or the androgen receptor, there is a need to develop novel therapeutic approaches to more effectively treat prostate, uterine, ovarian, and breast tumors and to overcome resistance to currently available hormone therapies.

### SUMMARY

This summary is provided to introduce a selection of concepts in a simplified form that are further described below in the Detailed Description. This summary is not intended to identify key features of the claimed subject matter, nor is it intended to be used as an aid in determining the scope of the claimed subject matter.

In yet another embodiment, the present disclosure provides a method of predicting earlier disease progression in a subject suffering from prostate cancer. In some embodiments, the method comprising: obtaining one or more biological samples from the subject; measuring the expression or DNA methylation level of at least one biomarker gene comprising PRAC1 in the one or more biological samples; comparing the expression or DNA methylation levels of the at least one biomarker gene comprising PRAC1 in the one or more biological sample obtained from the subject with an expression or DNA methylation level of PRAC1 in a reference/control sample. In some embodiments, a lower expression or higher DNA methylation level of PRAC1 (PRAC1^{low}) in the one or more biological sample obtained from the subject relative to the reference/control sample predicts an earlier disease progression in the subject. In some embodiments, the subject is resistant to androgen deprivation therapy. In some embodiments, the estrogen driven cancer is estrogen receptor positive.

### DESCRIPTION OF THE DRAWINGS

The foregoing aspects and many of the attendant advantages of this invention will become more readily appreciated as the same become better understood by reference to the following detailed description, when taken in conjunction with the accompanying drawings, wherein:

FIG. 1A depicts an analysis of publicly available RNA-sequencing data showing an association with PRAC1 expression with the response to the ARSI enzalutamide. FIG. 1B shows that PRAC1 expression is tightly correlated with PRAC1 gene locus methylation.

FIGS. 2A and 2B show the reading frame of PRAC1 translated in cancer. FIG. 2A shows that the first open reading frame of PRAC1 is translated in prostate cancer. FIG. 2B shows that expression of the first, but not the second open reading frame results in growth suppression of castration resistant prostate cancer models.

FIG. 3 shows expression changes associated with PRAC1 re-expression. The expression level changes demonstrate that PRAC1 is regulating prostate cancer cell differentiation and proliferation.

FIG. 4 shows that PRAC1 regulates sensitivity to AR-directed therapies. (PRAC1; expressed from a lentiviral construct with expressing ORF1 SEQ ID NO:2) in combination with enzalutamide, black line) in cell lines (shown here are LNCaP-abl cells) that are resistant to the AR signaling inhibitors (ARSI) enzalutamide results in restoration of sensitivity to this drug. Cells expressing control vectors (RFP) are resistant to enzalutamide across a broad concentration range.

FIGS. 5A to 5D show that PRAC1 mimetic peptides suppress cancer growth. FIG. 5A depicts the growth suppressive activity in a dose dependent manner of overlapping 10-mer PRAC1 peptide fragments in LNCaP-abl cells which were synthesized and conjugated to the arginine rich regions of TAT (RKKRRQRRRG; SEQ ID NO:7). FIG. 5B demonstrates the uptake of the PRAC1 C-terminal (LFSLIRKHLLRKKRRQRRRG; SEQ ID NO:17) labeled at the N-terminus with FITC in LNCaP-abl cells. (FIG. 5C) shows the growth and cell viability suppressive activity of PRAC1 peptide (PRAC1-F6 (SEQ ID NO:17)) by androgen receptor positive cell lines LNCaP and LNCaP-abl and AR-negative cell lines PC3 and DU145. Only AR-expressing cell lines show profound dose-dependent decrease in cell viability. FIG. 5D shows in vivo delivery of PRAC1-F6 peptide (SEQ ID NO:17) results in suppression of LAPC4-CR tumor growth.

FIGS. 6A to 6C show the structural requirements for AR-PRAC1 interaction. FIG. 6A is an AlphaFold model showing the interaction of PRAC1 with the androgen receptor (AR). FIG. 6B shows crystal structure analyses of the AR ligand binding domain bound to PRAC1 peptide LFSLIRKHLL (SEQ ID NO:37). FIG. 6C shows using a recombinant protein-based assay that PRAC1 specifically binds the AR LBD even under highly stringent conditions (0.5 M NaCl).

FIGS. 7A to 7C show the systematic analysis of crucial amino acid residues required for PRAC1 function. FIG. 7A depicts the effect of PRAC1 peptide alanine substitution of cell viability in LNCaP-abl cells. While active peptide PPRVLFSLIRKHLLRKKRRQRRG (SEQ ID NO:18) exhibited profound anti-tumor cell activity, an isoleucine-to-alanine mutant peptide (19A) PPRVLFSLARKHLLRKKQRRRG (SEQ ID NO:27) showed no effect on cell viability, and an arginine-to-alanine mutant peptide (R11A) PPRVLFSLIAKHLLRKKQRRRG (SEQ ID NO:28) showed attenuated activity on cell viability. FIG. 7B depicts the crystal structure of the AR LBD bound to PRAC1 that was resolved for the arginine reach region containing PRAC1 peptide PRVLFSLIRKHLLRKKRRQ (amino acid residues 2 to 20 of SEQ ID NO:34) and the network of interactions between the two proteins is identical to those observed in the crystal structure of the AR LBD bound to PRAC1 peptide LFSLIRKHLL (SEQ ID NO:37). FIG. 7C shows the cell viability of LNCaP-abl cells exposed to different PRAC1 peptides. The peptides tested include LFSLIRKHLLRKKRRQRRRG (SEQ ID NO:17), PPRVLFSLIRKHLLRKKQRRRG (SEQ ID NO:18), PPRVLFSLIRKHLLRKK (SEQ ID NO:33), PPRVLFSLIRKHLLRKKRRQ (SEQ ID NO:34), FSLIRKHLLRKKR (SEQ ID NO:35), FSLIRKHLLRKKRRQ (SEQ ID NO:36), and a scrambled control peptide.

FIGS. 8A and 8B show PRAC1 peptide activity in estrogen receptor positive breast cancer. FIG. 8A show the viability of estrogen receptor positive MCF7 breast cancer cells cultured with the PRAC1 peptide PPRVLFSLIRKHLLRKKRRQRRRG (SEQ ID NO:18). Cells treated with the PRAC1 peptide demonstrated a dose-dependent decrease is cell viability relative to the scrambled control peptide. FIG. 8B shows an AlphaFold model demonstrating the interaction of full length PRAC1 (SEQ ID NO:3) to the estrogen receptor alpha.

FIG. 9 shows representative TR-FRET data from a scalable high throughput assay to study AR-PRAC1 interactions.

FIGS. 10A to 10C provide an overview of the PRAC1 stapled and cyclic peptide sequences and structure-based design strategy for the i, i+7 PRAC1 peptide-based crystal structures or the AR LBD bound to PRAC1 peptides. The stapled PRAC1 peptides were designed to stabilize and extend the AR-interacting region (FIG. 10A). Cyclic peptides were designed with an alpha-helix core based on the crystal structures in bold and underlined flanked by disordered loops to mediate head to tail amide cyclization (FIG. 10B). FIG. 10C is an AlphaFold model of the i, i+7 stapled peptide structure bound to AR.

### DETAILED DESCRIPTION

The following pages disclose aspects and embodiments related to targeting PRAC1 in steroid hormone driven cancers.

One skilled in the art will recognize many methods and materials similar or equivalent to those described herein, which could be used in and are within the scope of the practice of the present invention. The present invention is in no way limited to the methods and materials described.

Unless defined otherwise, technical, and scientific terms used herein have the same meaning as commonly understood by one of ordinary skill in the art to which this invention belongs. Although any methods, devices, and materials similar or equivalent to those described herein can be used in the practice or testing of the invention, the preferred methods, devices, and materials are now described.

All publications, published patent documents, and patent applications cited in this application are indicative of the level of skill in the art(s) to which the application pertains. All publications, published patent documents, and patent applications cited herein are hereby incorporated by reference to the same extent as though each individual publication, published patent document, or patent application was specifically and individually indicated as being incorporated by reference.

As used in this application, including the appended claims, the singular forms “a,” “an,” and “the” include plural references, unless the content clearly dictates otherwise, and are used interchangeably with “at least one” and “one or more.” Thus, reference to “an aptamer” includes mixtures of aptamers, reference to “a probe” includes mixtures of probes, and the like.

As used herein, the terms “comprises,” “comprising,” “includes,” “including,” “contains,” “containing,” and any variations thereof, are intended to cover a non-exclusive inclusion, such that a process, method, product-by-process, or composition of matter that comprises, includes, or contains an element or list of elements does not include only those elements but may include other elements not expressly listed or inherent to such process, method, product-by-process, or composition of matter.

The term “consisting essentially of” limits the scope of a claim to the specified materials or steps, or to those that do not materially affect the basic characteristics of the claimed subject matter. For example, a protein domain, region, or module (e.g., a binding domain, hinge region, linker module) or a protein (which may have one or more domains, regions, or modules) “consists essentially of” a particular amino acid sequence when the amino acid sequence of a domain, region, module, or protein includes extensions, deletions, mutations, or a combination thereof (e.g., amino acids at the amino- or carboxy-terminus or between domains) that, in combination, contribute to at most 20% (e.g., at most 15%, 10%, 8%, 6%, 5%, 4%, 3%, 2% or 1%) of the length of a domain, region, module, or protein and do not substantially affect (i.e., do not reduce the activity by more than 50%, such as no more than 40%, 30%, 25%, 20%, 15%, 10%, 5%, or 1%) the activity of the domain(s), region(s), module(s), or protein (e.g., the target binding affinity of a binding protein).

In addition, it should be understood that the individual features or groups of features, derived from the various combinations of the compositions and substituents described herein, are disclosed by the present application to the same extent as if each feature or group of features was set forth individually. Thus, selection of particular structures or particular substituents is within the scope of the present disclosure.

“Biological sample”, “sample”, and “test sample” are used interchangeably herein to refer to any material, biological fluid, tissue, or cell obtained or otherwise derived from an individual. This includes blood (including whole blood, leukocytes, peripheral blood mononuclear cells, buffy coat, plasma, and serum), sputum, tears, mucus, nasal washes, nasal aspirate, breath, urine, semen, saliva, peritoneal washings, ascites, cystic fluid, meningeal fluid, amniotic fluid, glandular fluid, pancreatic fluid, lymph fluid, pleural fluid, nipple aspirate, bronchial aspirate, bronchial brushing, synovial fluid, joint aspirate, organ secretions, cells, a cellular extract, and cerebrospinal fluid. This also includes experimentally separated fractions of all the preceding. For example, a blood sample can be fractionated into serum, plasma or into fractions containing particular types of blood cells, such as red blood cells or white blood cells (leukocytes). If desired, a sample can be a combination of samples from an individual, such as a combination of a tissue and fluid sample. The term “biological sample” also includes materials containing homogenized solid material, such as from a stool sample, a tissue sample, or a tissue biopsy, for example. The term “biological sample” also includes materials derived from a tissue culture or a cell culture. Any suitable methods for obtaining a biological sample can be employed; exemplary methods include, e.g., phlebotomy, swab (e.g., buccal swab), and a fine needle aspirate biopsy procedure. Exemplary tissues susceptible to fine needle aspiration include lymph node, lung, lung washes, BAL (bronchoalveolar lavage), thyroid, breast, pancreas, and liver. Samples can also be collected, e.g., by micro dissection (e.g., laser capture micro dissection (LCM) or laser micro dissection (LMD)), bladder wash, smear (e.g., a PAP smear), or ductal lavage. A “biological sample” obtained or derived from an individual includes any such sample that has been processed in any suitable manner after being obtained from the individual. “Biological sample” includes sections of tissues such as biopsy and autopsy samples, and frozen sections taken for histologic purposes. Such samples include prostate cancer tissues, cultured cells, e.g., primary cultures, explants, and transformed cells. A biological sample is typically obtained from a mammal, such as a primate, e.g., human. The biological sample, in some embodiments, may include metastatic tissue. It would be readily understood by those skilled in the art that expression levels of biomarkers disclosed herein could be measured in biological samples such as tissue samples obtained by fine needle aspiration (FNAB) (a preferred embodiment), formalin-fixed paraffin embedded (FFPE) tissue, tissue microarrays (TMA), fresh-frozen or freshly obtained prostate cancer biopsy material.

A “biopsy” refers to the process of removing a tissue sample for diagnostic or prognostic evaluation, and to the tissue specimen itself. Any biopsy technique known in the art can be applied to the diagnostic and prognostic methods. The biopsy technique applied will depend on the tissue type to be evaluated, the size and type of the tumor, among other factors. Representative biopsy techniques include, but are not limited to, excisional biopsy, incisional biopsy, needle biopsy, and surgical biopsy. An “excisional biopsy” refers to the removal of an entire tumor mass with a small margin of normal tissue surrounding it. An “incisional biopsy” refers to the removal of a wedge of tissue that includes a cross-sectional diameter of the tumor. A diagnosis or prognosis made by endoscopy or fluoroscopy can require a “core-needle biopsy”, or a “fine-needle aspiration biopsy” which generally obtains a suspension of cells from within a target tissue. Biopsy techniques are discussed, for example, in Harrison's Principles of Internal Medicine, 2005. Obtaining a biopsy includes both direct and indirect methods, including obtaining the biopsy from the patient or obtaining the biopsy sample after it is removed from the patient.

As used herein, “marker” and “biomarker” are used interchangeably to refer to a target molecule that indicates or is a sign of a normal or abnormal process in an individual/subject or of a disease or other condition in an individual/subject. More specifically, a “marker” or “biomarker” is an anatomic, physiologic, biochemical, or molecular parameter associated with the presence of a specific physiological state or process, whether normal or abnormal, and, if abnormal, whether chronic or acute. Biomarkers are detectable and measurable by a variety of methods including laboratory assays and medical imaging. When a biomarker is a protein, it is also possible to use the expression of the corresponding gene as a surrogate measure of the amount or presence or absence of the corresponding protein biomarker in a biological sample or methylation state of the gene encoding the biomarker or proteins that control expression of the biomarker. A biomarker or a biomarker panel is a measured characteristic, substance, or analyte or group of characteristics, substances or analytes that are objectively measured and evaluated as an indicator of normal biological processes, pathogenic processes, or pharmacologic responses to a therapeutic intervention. Cancer staging, including identification and/or localization of tumor, nodes, and or metastases (TNM), may be the broadest clinical set of biomarkers. Biomarkers can be readily attained from patient samples for routine monitoring and thus biomarkers analyzed in whole blood, serum or plasma, urine, mucous, feces, tears, semen, and the like are most easily obtainable. However, in some cases, biomarkers may be analyzed in patient samples that require more invasive procedures such as biopsy or tissue sampling for example tumor, bone, skin, teeth, organ biopsy (liver, kidney, colon, lung pancreas). Alternatively, circulating tumor cells or exosomes from prostate tumor or metastatic cells may be tested for biomarkers. Biomarkers include biological, physiological molecules, compounds, substances, or analytes and are analyzed to determine an absence/presence, level, concentration, value, intensity, activity, or measurement.

As used herein, “biomarker value”, “value”, “biomarker level”, and “level” are used interchangeably to refer to a measurement that is made using any analytical method for detecting the biomarker in a biological sample and that indicates the presence, absence, absolute amount or concentration, relative amount or concentration, titer, a level, an expression level, a ratio of measured levels, or the like, of, for, or corresponding to the biomarker in the biological sample. The terms “level” or “levels” with reference to one or more biomarker is meant to encompass a score, quantitative measurement, a qualitative assessment, or other acceptable observation obtained when a biomarker or observation correlated to a biomarker is assessed. The exact nature of the “value” or “level” depends on the specific design and components of the particular analytical method employed to detect the biomarker.

The terms “overexpress”, “overexpression”, “overexpressed”, “up-regulate”, or “up-regulated” interchangeably refer to a biomarker that is transcribed or translated at a detectably greater level, usually in a cancer cell, in comparison or relative to a non-cancer cell or cancer cell (from a control or reference sample) that is not associated with the worst or poorest prognosis. The term includes overexpression due to transcription, post transcriptional processing, translation, post-translational processing, cellular localization, and/or RNA and protein stability, as compared to a non-cancer cell or cancer cell that is not associated with the worst or poorest prognosis. Overexpression can be detected using conventional techniques for detecting mRNA (i.e., RT-PCR, PCR, hybridization, RNA-sequencing, and the like) or proteins (i.e., ELISA, immunohistochemical techniques, mass spectroscopy, and the like). Overexpression can be 10%, 20%, 30%, 40%, 50%, 60%, 70%, 80%, 90% or more (or any range derivable therein) in comparison to a reference/control sample, normal cell or cancer cell that is not associated with the worst or poorest prognosis. In certain instances, overexpression is 1-fold, 2-fold, 3-fold, 4-fold 5, 6, 7, 8, 9, 10, or 15-fold or more higher levels of transcription or translation (or any range derivable therein) in comparison to or relative to a reference/control sample, non-cancer cell or cancer cell that is not associated with the worst or poorest prognosis.

The term “lower expression” or “low expression” includes a lower expression of the biomarker due to transcription, post transcriptional processing, translation, post-translational processing, cellular localization, and/or RNA and protein stability, as compared to a non-cancer cell or cancer cell that is not associated with the worst or poorest prognosis. A lower expression can be detected using conventional techniques for detecting mRNA (i.e., RT-PCR, PCR, hybridization, RNA-sequencing, and the like) or proteins (i.e., ELISA, immunohistochemical techniques, mass spectroscopy, and the like). A lower expression can be 10%, 20%, 30%, 40%, 50%, 60%, 70%, 80%, 90% or more (or any range derivable therein) in comparison to a reference/control sample, normal cell or cancer cell that is not associated with the worst or poorest prognosis. In certain instances, lower expression is 1-fold, 2-fold, 3-fold, 4-fold 5, 6, 7, 8, 9, 10, or 15-fold or more lower levels of transcription or translation (or any range derivable therein) in comparison/relative to a reference/control sample, non-cancer cell or cancer cell that is not associated with the worst or poorest prognosis.

As used herein, “increased expression” or “elevated expression” or “decreased expression” refers to an expression level of a biomarker in the subject's sample as compared to a reference level representing the same biomarker or a different biomarker. In certain aspects, the reference level may be a reference level of expression from a non-cancerous tissue from the same subject. Alternatively, the reference level may be a reference level of expression from a different subject or group of subjects. For example, the reference level of expression may be an expression level obtained from a sample (e.g., a tissue, fluid, or cell sample) of a subject or group of subjects without cancer, or an expression level obtained from a non-cancerous tissue of a subject or group of subjects with cancer. The reference level may be a single value or may be a range of values. The reference level of expression can be determined using any method known to those of ordinary skill in the art. In some embodiments, the reference level is an average level of expression determined from a cohort of subjects with cancer or without cancer. The reference level may also be depicted graphically as an area on a graph. In certain embodiments, a reference level is a normalized level.

The comparison may be a direct comparison where the expression level of a control is measured at the same time as the biological sample obtained from the subject or it may be a level of expression that is determined from a previously evaluated sample or an average of levels of expression of previously evaluated sample(s). Further, a biomarker that is either over-expressed or under-expressed can also be referred to as being “differentially expressed” or as having a “differential level” or “differential value” as compared to a “normal” expression level or value of the biomarker that indicates or is a sign of a normal process or an absence of a disease or other condition in an individual. Thus, “differential expression” of a biomarker can also be referred to as a variation from a “normal” expression level of the biomarker.

The term “differential gene expression” and “differential expression” are used interchangeably to refer to a gene (or its corresponding protein expression product) whose expression is activated to a higher or lower level in a subject suffering from a specific disease, relative to its expression in a normal or control subject and/or in a biological sample from a normal or control subject. The terms also include genes (or the corresponding protein expression products) whose expression is activated to a higher or lower level at different stages of the same disease. It is also understood that a differentially expressed gene may be either activated or inhibited at the nucleic acid level or protein level or may be subject to alternative splicing to result in a different polypeptide product. Such differences may be evidenced by a variety of changes including mRNA levels, surface expression, secretion, or other partitioning of a polypeptide. Differential gene expression may include a comparison of expression between two or more genes or their gene products; or a comparison of the ratios of the expression between two or more genes or their gene products; or even a comparison of two differently processed products of the same gene, which differ between normal subjects and subjects suffering from a disease; or between various stages of the same disease. Differential expression includes both quantitative, as well as qualitative, differences in the temporal or cellular expression pattern in a gene or its expression products among, for example, normal and diseased cells, or among cells which have undergone different disease events or disease stages.

As used herein, “subject” or “individual” or “patient”, refers to a test subject or patient. The subject can be a mammal or a non-mammal. In various embodiments, the subject is a mammal. A mammalian subject can be a human or non-human. In various embodiments, the subject is a human. A healthy or normal subject is an individual in which the disease or condition of interest (including, for example, prostate cancer, castration resistant prostate cancer) is not detectable by conventional diagnostic methods.

“Diagnose”, “diagnosing”, “diagnosis”, and variations thereof refer to the detection, determination, or recognition of a health status or condition of an individual or subject on the basis of one or more signs, symptoms, data, or other information pertaining to that individual. The health status of an individual or subject can be diagnosed as healthy/normal (i.e., a diagnosis of the absence of a disease or condition) or diagnosed as ill/abnormal (i.e., a diagnosis of the presence, or an assessment of the characteristics, of a disease or condition). The terms “diagnose”, “diagnosing”, “diagnosis”, and the like, encompass, with respect to a particular disease or condition, the initial detection of the disease; the characterization or classification of the disease; the detection of the progression, remission, or recurrence of the disease; and the detection of disease response after the administration of a treatment or therapy to the individual.

“Prediction” as used herein refers to a prediction of whether an individual is predisposed to or will develop a steroid hormone driven cancer. Early determination of predisposition or likelihood of developing a cancer can provide for earlier treatment of the cancer and a better likelihood of survival.

“Prognosis” generally refers to a prediction of the probable course or outcome of the disease. As used herein, prognosis includes the forecast or prediction of any one or more of the following: duration of survival of a subject susceptible to or diagnosed with a cancer or disease, duration of recurrence-free survival, duration of progression free survival of a subject susceptible to or diagnosed with a cancer, response rate in a group of patients susceptible to or diagnosed with a cancer, duration of response in a patient or a group of patients susceptible to or diagnosed with a cancer, and/or likelihood of metastasis in a patient susceptible to or diagnosed with a cancer. As used herein, “prognostic for cancer” means providing a forecast or prediction of the probable course or outcome of the cancer. In some embodiments, “prognostic for cancer” comprises providing the forecast or prediction of (prognostic for) any one or more of the following: duration of survival of a patient susceptible to or diagnosed with a cancer, duration of recurrence-free survival, duration of progression free survival of a patient susceptible to or diagnosed with a cancer, response rate in a group of patients susceptible to or diagnosed with a cancer, duration of response in a patient or a group of patients susceptible to or diagnosed with a cancer, and/or likelihood of metastasis in a patient susceptible to or diagnosed with a cancer.

The term “substantially the same” or “not significantly different” refers to a level of expression that is not significantly different than what it is compared to. Alternatively, or in conjunction, the term substantially the same refers to a level of expression that is less than 2, 1.5, or 1.25-fold different than the expression or activity level it is compared to.

The term “disease free survival” is a clinical endpoint and is usually used to analyze the results of the treatment for the localized disease which renders the patient apparently disease free, such as surgery or surgery plus adjuvant therapy. In the disease-free survival, the event is relapse rather than death. The people who relapse are still surviving but they are no longer disease-free. Just as in the survival curves not all patients die, in “disease-free survival curves” not all patients relapse, and the curve may have a final plateau representing the patients who didn't relapse after the study's maximum follow-up. Because the patients survive for at least some time after the relapse, the curve for the actual survival would look better than disease free survival curve.

“About” and “approximately” shall generally mean an acceptable degree of error for the quantity measured given the nature or precision of the measurements. Typically, exemplary degrees of error are within 20 percent (%), preferably within 10%, and more preferably within 5% of a given value or range of values. Alternatively, and particularly in biological systems, the terms “about” and “approximately” may mean values that are within an order of magnitude, preferably within 5-fold and more preferably within 2-fold of a given value. In some embodiments it is contemplated that a numerical value discussed herein may be used with the term “about” or “approximately.”

The term “polypeptide” or “peptide” is used interchangeably and encompasses two or more naturally or non-naturally occurring amino acids (e.g., most typically L-amino acids, but also including, e.g., D-amino acids, modified amino acids, amino acid analogs, and amino acid mimetic) joined by a covalent bond (e.g., an amide bond). Polypeptides as described herein include full length proteins (e.g., fully processed proteins) as well as shorter amino acid sequences (e.g., fragments or derivatives of naturally occurring proteins or synthetic polypeptide fragments or peptides comprising one or more modified amino acids). For purposes of the present disclosure, a “peptide” is not limited to any particular number of amino acids. Preferably, however, the peptide contains about 2 to about 50 amino acids (e.g., about 5 to about 40 amino acids, about 5 to about 20 amino acids, or about 7 to 15 amino acids). The terms “peptide,” “polypeptide,” and “protein” are used interchangeably herein and typically refer to a molecule comprising a chain of two or more amino acids.

The terms “polypeptide” and “peptide” as used herein refer to a compound made up of amino acid residues that are linked by peptide bonds. The term “protein” may be synonymous with the term “polypeptide” or may refer, in addition, to a complex of two or more polypeptides. A polypeptide may further contain other components (e.g., covalently bound), such as a tag, a label, a bioactive molecule, or any combination thereof. In certain embodiments, a polypeptide may be a fragment or derivative. As used herein, a “fragment” or “derivative” means a polypeptide that is lacking one or more amino acids that are found in a parent polypeptide.

In certain embodiments, a derivative of a polypeptide can have at least about 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99%, or more of amino acids of the amino acid sequence of the parent polypeptide.

Peptides may be naturally occurring, synthetically produced, or recombinantly expressed. Peptides may also comprise additional groups modifying the amino acid chain, for example, functional groups added via post-translational modification. Examples of post-translation modifications include, but are not limited to, acetylation, alkylation (including, methylation), biotinylation, glutamylation, glycylation, glycosylation, isoprenylation, lipoylation, phosphopantetheinylation, phosphorylation, selenation, and C-terminal amidation. The term peptide also includes peptides comprising modifications of the amino terminus and/or the carboxy terminus. Modifications of the terminal amino group include, but are not limited to, des-amino, N-lower alkyl, N-di-lower alkyl, and N-acyl modifications. Modifications of the terminal carboxy group include, but are not limited to, amide, lower alkyl amide, dialkyl amide, and lower alkyl ester modifications (e.g., wherein lower alkyl is C₁-C₄ alkyl). The term peptide also includes modifications, such as but not limited to those described above, of amino acids falling between the amino and carboxy termini. In one embodiment, a peptide may be modified by addition of a small-molecule drug.

The terminal amino acid at one end of the peptide chain typically has a free amino group (i.e., the amino terminus). The terminal amino acid at the other end of the chain typically has a free carboxyl group (i.e., the carboxy terminus). Typically, the amino acids making up a peptide are numbered in order, starting at the amino terminus and increasing in the direction of the carboxy terminus of the peptide.

The phrase “amino acid residue” as used herein refers to an amino acid that is incorporated into a peptide by an amide bond or an amide bond mimetic.

The term “amino acid” is used to mean an organic compound containing an amino group and a carboxyl group and to mean an α-amino acid contained as a constituent unit preferably in a protein, and more preferably in a natural protein. The term “naturally occurring amino acid” refers to any one of the twenty amino acids commonly found in peptides synthesized in nature, and known by the one letter abbreviations A, R, N, C, D, Q, E, G, H, I, L, K, M, F, P, S, T, W, Y and V. The term “amino acid” is used to mean these 20 amino acids in total, unless otherwise specified. These 20 amino acids in total can be referred to as “natural amino acids”. The term “amino acid residue” is also referred to as an “amino acid”. Naturally occurring amino acids are those encoded by the genetic code, as well as those amino acids that are later modified, e.g., hydroxyproline, γ-carboxyglutamate, and O-phosphoserine. The amino acid may be an L-amino acid, a D-amino acid, or a mixture thereof (DL-amino acid) and means an L-amino acid, unless otherwise specified. Natural amino acids can be divided into, for example, the following groups, on the basis of the common properties of side chains: (1) hydrophobic amino acid group: Met, Ala, Val, Leu, and Ile; (2) neutral hydrophilic amino acid group: Cys, Ser, Thr, Asn, and Gln; (3) acidic amino acid group: Asp and Glu; (4) basic amino acid group: His, Lys, and Arg; (5) group of amino acids influencing the direction of the main chain: Gly and Pro; and (6) aromatic amino acid group: Trp, Tyr, and Phe.

In the present disclosure an amino acid may be an amino acid other than natural amino acids. Examples thereof can include synthetic amino acids, as well as amino acid analogs and amino acid mimetics that function in a manner similar to the naturally occurring amino acids. Amino acid analogs refers to compounds that have the same basic chemical structure as a naturally occurring amino acid, i.e., a carbon that is bound to a hydrogen, a carboxyl group, an amino group, and an R group, e.g., homoserine, norleucine, methionine sulfoxide, methionine methyl sulfonium, R-2-(7′-octenyl) alanine, S-2-(4′-pentenyl) alanine, and the like. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid.

The term “α-amino acid” or simply “amino acid” refers to a molecule containing both an amino group and a carboxyl group bound to a carbon which is designated the α-carbon. Suitable amino acids include, without limitation, both the D- and L-isomers of the naturally-occurring amino acids, as well as non-naturally occurring amino acids prepared by organic synthesis or other metabolic routes. Unless the context specifically indicates otherwise, the term amino acid, as used herein, is intended to include amino acid analogs.

The term “amino acid analog” or “non-natural amino acid” refers to a molecule which is structurally similar to an amino acid, and which can be substituted for an amino acid in the formation of a peptidomimetic macrocycle. Amino acid analogs include, without limitation, compounds which are structurally identical to an amino acid, as defined herein, except for the inclusion of one or more additional methylene groups between the amino and carboxyl group (e.g., α-amino (3-carboxy acids), or for the substitution of the amino or carboxy group by a similarly reactive group (e.g., substitution of the primary amine with a secondary or tertiary amine, or substitution or the carboxy group with an ester).

A “non-essential” amino acid residue is a residue that can be altered from the wild-type sequence of a polypeptide (e.g., a BH3 domain or the p53 MDM2 binding domain) without abolishing or substantially altering its essential biological or biochemical activity (e.g., receptor binding or activation). An “essential” amino acid residue is a residue that, when altered from the wild-type sequence of the polypeptide, results in abolishing or substantially abolishing the polypeptide's essential biological or biochemical activity.

In the present disclosure, each natural amino acid may receive a conservative amino acid substitution.

A “conservative amino acid substitution” is one in which the amino acid residue is replaced with an amino acid residue having a similar side chain. Families of amino acid residues having similar side chains have been defined in the art. These families include amino acids with basic side chains (e.g., K, R, H), acidic side chains (e.g., D, E), uncharged polar side chains (e.g., G, N, Q, S, T, Y, C), nonpolar side chains (e.g., A, V, L, I, P, F, M, W), beta-branched side chains (e.g., T, V, I) and aromatic side chains (e.g., Y, F, W, H). Thus, a predicted nonessential amino acid residue in a BH3 polypeptide, for example, is preferably replaced with another amino acid residue from the same side chain family. Other examples of acceptable substitutions are substitutions based on isosteric considerations (e.g., norleucine for methionine) or other properties (e.g., 2-thienylalanine for phenylalanine).

The conservative amino acid substitution includes, as shown below, a specific substitution based on side chain similarity (L. Lehninger, Biochemistry, 2nd edition, pp. 73-75, Worth Publisher, New York (1975) and a typical substitution. (1) Nonpolar amino acid group: alanine (hereinafter, referred to as “Ala” or simply “A”), valine (hereinafter, referred to as “Val” or simply “V”), leucine (hereinafter, referred to as “Leu” or simply “L”), isoleucine (hereinafter, referred to as “11e” or simply “T”), proline (hereinafter, referred to as “Pro” or simply “P”), phenylalanine (hereinafter, referred to as “Phe” or simply “F”), tryptophan (hereinafter, referred to as “Trp” or simply “W”), and methionine (hereinafter, referred to as “Met” or simply “M”); (2) Uncharged polar amino acid group: glycine (hereinafter, referred to as “Gly” or simply “G”), serine (hereinafter, referred to as “Ser” or simply “S”), threonine (hereinafter, referred to as “Thr” or simply “T”), cysteine (hereinafter, referred to as “Cys” or simply “C”), tyrosine (hereinafter, referred to as “Tyr” or simply “Y”), asparagine (hereinafter, referred to as “Asn” or simply “N”), and glutamine (hereinafter, referred to as “Gin” or simply “Q”); (3) Acidic amino acid group: aspartic acid (hereinafter, referred to as “Asp” or simply “D”) and glutamic acid (hereinafter, referred as to “Glu” or simply “E”) (4) Basic amino acid group: lysine (hereinafter, referred to as “Lys” or simply “K”), arginine (hereinafter, referred to as “Arg” or simply “R”), and histidine (hereinafter, referred to as “His” or simply “H”).

In the present disclosure, an isolated peptide prepared by the substitution, addition and/or deletion of amino acids in, to or from the PRAC 1 peptide or the N-terminal and/or C-terminal of the PRAC1 peptide (hereinafter, referred to as a “parent peptide”) is referred to as a “derivative”. The number of amino acid residues substituted, added, and/or deleted can be as few as one or two amino acids, or can be as many as 10, 20, 30, 40 or more amino acid residues. Such a “derivative” is also included in the scope of the “peptide” or “polypeptide” of the present disclosure.

The term “member” as used herein in conjunction with macrocycles or macrocycle-forming linkers refers to the atoms that form or can form the macrocycle and excludes substituent or side chain atoms. By analogy, cyclodecane, 1,2-difluoro-decane and 1,3-dimethyl cyclodecane are all considered ten-membered macrocycles as the hydrogen, or fluoro substituents or methyl side chains do not participate in forming the macrocycle.

The term “amino acid side chain” refers to a moiety attached to the α-carbon in an amino acid. For example, the amino acid side chain for alanine is methyl, the amino acid side chain for phenylalanine is phenylmethyl, the amino acid side chain for cysteine is thiomethyl, the amino acid side chain for aspartate is carboxymethyl, the amino acid side chain for tyrosine is 4-hydroxyphenylmethyl, etc. Other non-naturally occurring amino acid side chains are also included, for example, those that occur in nature (e.g., an amino acid metabolite) or those that are made synthetically (e.g., an α,α di-substituted amino acid).

As used herein, the term “crosslinked polypeptide”, “cross-linked peptide”, “stapled peptide”, or “Peptidomimetic macrocycles”, refers to a compound comprising a plurality of amino acid residues joined by a plurality of peptide bonds and at least one macrocycle-forming linker which forms a macrocycle between a first naturally-occurring or non-naturally-occurring amino acid residue (or analog) and a second naturally-occurring or non-naturally-occurring amino acid residue (or analog) within the same molecule. Cross-linked peptides or Peptidomimetic macrocycles include embodiments where the macrocycle-forming linker connects the α carbon of the first amino acid residue (or analog) to the α carbon of the second amino acid residue (or analog).

Stapled peptides include embodiments where the macrocycle-forming linker connects the side chain of one amino acid residue (or analog, such as S-2-(4′-pentenyl) alanine, also referred to as S5) to the side chain of another amino acid residue (or analog, such as R-2-(7′-octenyl) alanine, also referred to as R8) on the same peptide to create a helical “brace.” These embodiments can include single “staples” with spacing (i, i+4; i, i+7) or double “staples” with the following spacing (I, i+4). Specific embodiments provided herein include the following:
PPXVLFSLIXKHLLRKKRRQRRRG, wherein X³ is R-2-(7′octanyl) alanine, and X¹⁰ is S-2-(4′-pentenyl) alanine (SEQ ID NO:38)
PPXVLFSLIXKHLL, wherein X³ is R-2-(7′octanyl) alanine, and X¹⁰ is S-2-(4′-pentenyl) alanine (SEQ ID NO:39)

The peptidomimetic macrocycles optionally include one or more non-peptide bonds between one or more amino acid residues and/or amino acid analog residues, and optionally include one or more non-naturally occurring amino acid residues or amino acid analog residues in addition to any which form the macrocycle.

One embodiment or form of macrocycle prepared in the present disclosure is a head-to-tail amide bond between the amino and carboxy terminus of certain peptide embodiments disclosed herein. Cyclization of peptides through head-to-tail synthesis results in a peptide that is non-linear, non-directional, and cyclical. Methods for forming head-to-tail amide cyclized peptides are well known in the art. Embodiments disclosed herein include addition of a disordered loop or ring to a PRAC1 α-helix core, wherein the disordered loop can be added to either the animo or carboxyl end of the PRAC1 peptide core. The disordered loop can be 5, 6, 7, 8, 9, or even more amino acids in length. The amino acids that comprise the disordered loop form a non-helical sequence of flexible, cell-permeable, and ring forming amino acid residues and can include, i.e., G, S, P, D, N, R, A, Q, C, H, Y, W, T, F, and K. Head-to-tail amide cyclic peptides can include the following:

(SEQ ID NO: 40)
XXXXXSLIRKHL
(SEQ ID NO: 41)
XXXXXSLIRKHLL
(SEQ ID NO: 42)
SLIRKHLXXXXX
(SEQ ID NO: 43)
SLIRKHLLXXXXX
(SEQ ID NO: 44)
XXXXXXSLIRKHL
(SEQ ID NO: 45)
XXXXXXSLIRKHLL
(SEQ ID NO: 46)
SLIRKHLXXXXXX
(SEQ ID NO: 47)
SLIRKHLLXXXXXX
(SEQ ID NO: 48)
XXXXXXXSLIRKHL
(SEQ ID NO: 49)
XXXXXXXSLIRKHLL
(SEQ ID NO: 50)
SLIRKHLXXXXXXX
(SEQ ID NO: 51)
SLIRKHLLXXXXXXX
(SEQ ID NO: 52)
XXXXXXXXSLIRKHL
(SEQ ID NO: 53)
XXXXXXXXSLIRKHLL
(SEQ ID NO: 54)
SLIRKHLXXXXXXXX
(SEQ ID NO: 55)
SLIRKHLLXXXXXXXX
(SEQ ID NO: 56)
XXXXXXXXXSLIRKHL
(SEQ ID NO: 57)
XXXXXXXXXSLIRKHLL
(SEQ ID NO: 58)
SLIRKHLXXXXXXXXX
(SEQ ID NO: 59)
SLIRKHLLXXXXXXXXX
(SEQ ID NO: 60)
SGPLGSLIRKHL
(SEQ ID NO: 61)
SLIRKHLSGPLG
(SEQ ID NO: 62)
SGPLGSLIRKHLL
(SEQ ID NO: 63)
SLIRKHLLSGPLG
(SEQ ID NO: 64)
XXXXXXLFSLIRKHLL
(SEQ ID NO: 65)
XXXXXXXLFSLIRKHLL
(SEQ ID NO: 66)
XXXXXXXXLFSLIRKHLL
(SEQ ID NO: 67)
XXXXXXXXXLFSLIRKHLL
(SEQ ID NO: 68)
LFSLIRKHLLXXXXXX
(SEQ ID NO: 69)
LFSLIRKHLLXXXXXXX
(SEQ ID NO: 70)
LFSLIRKHLLXXXXXXXX
(SEQ ID NO: 71)
LFSLIRKHLLXXXXXXXXX
(SEQ ID NO: 72)
SGPLGLFSLIRKHLL
(SEQ ID NO: 73)
LFSLIRKHLLSGPLG

The term “gene” is used to mean a nucleic acid molecule comprising a nucleotide sequence encoding an amino acid sequence contained in a protein, or a complementary strand thereof. The gene consists of a single strand, a double strand, or a triple or more strand. An association of a DNA strand and an RNA strand, ribonucleotides and deoxyribonucleotides coexisting on one strand, and a double-stranded or triple- or more stranded nucleic acid molecule including such a strand are also included in the meaning of the “gene.”

A “nucleic acid molecule” or “polynucleotide” refers to a single- or double-stranded linear or circular polynucleotide containing either deoxyribonucleotides or ribonucleotides that are linked by 3′-5′-phosphodiester bonds. A nucleic acid molecule includes RNA, DNA, genomic DNA, mitochondrial DNA, cDNA, or vector DNA. In certain embodiments, the nucleic acids of the present disclosure are produced by PCR. Nucleic acids may be composed of monomers that are naturally occurring nucleotides (such as deoxyribonucleotides and ribonucleotides), analogs of naturally occurring nucleotides (e.g., a-enantiomeric forms of naturally-occurring nucleotides, morpholinos), or a combination of both. Modified nucleotides can have modifications in, or replacement of, sugar moieties, or pyrimidine or purine base moieties. Nucleic acid monomers can be linked by phosphodiester bonds or analogs of such linkages. Analogs of phosphodiester linkages include phosphorothioate, phosphorodithioate, phosphoroselenoate, phosphorodiselenoate, phosphoroanilothioate, phosphoranilidate, phosphoramidate, and the like.

In certain embodiments, polynucleotides encoding peptides or proteins of the disclosure may be codon optimized to enhance or maximize expression in certain types of cells (e.g., Scholten et al., Clin. Immunol. 119:135-145, 2006). As used herein a “codon optimized” polynucleotide is a heterologous polypeptide having codons modified with silent mutations corresponding to the abundances of host cell tRNA levels.

Variants of the polynucleotides of this disclosure are also contemplated. Variant polynucleotides are at least 80%, 85%, 90%, 95%, 99%, or 99.9% identical to a reference polynucleotide as described herein.

“Expression vector,” as used herein, refers broadly to any recombinant expression system for the purpose of expressing a nucleic acid sequence of the disclosure in vitro or in vivo, constitutively or inducibly, in any cell, including prokaryotic, yeast, fungal, plant, insect or mammalian cell. The term includes linear or circular expression systems. The term includes expression systems that remain episomal or that integrate into the host cell genome. The expression systems can have the ability to self-replicate or not, i.e., drive only transient expression in a cell. The term includes recombinant expression cassettes which contain only the minimum elements needed for transcription of the recombinant nucleic acid.

The term “expression vector” or “vector” is to be broadly interpreted as including a plasmid, including an episome, a viral vector, a cosmid, or the like. A vector can be single-stranded or double-stranded, and can comprise RNA, DNA, or modifications and combinations thereof. Selection of a vector or plasmid backbone depends upon a variety of characteristics desired in the resulting construct, such as selection marker(s), plasmid copy number, and the like. A nucleic acid sequence is “operably linked” to an expression control sequence in the regulatory region of a vector, such as a promoter, when the expression control sequence controls or regulates the transcription and/or the translation of that nucleic acid sequence. A nucleic acid that is “operably linked” to an expression control sequence includes, for example, an appropriate start signal (e.g., ATG) at the beginning of the nucleic acid sequence to be expressed and a reading frame that permits expression of the nucleic acid sequence under control of the expression control sequence to yield production of the encoded peptide. The regulatory region of the expression vector optionally includes a termination sequence, such as a codon for which there is no corresponding aminoacetyl-tRNA, thus ending peptide synthesis. Typically, when the ribosome reaches a termination sequence or codon during translation of the mRNA, the polypeptide is released and the ribosome-mRNA-tRNA complex dissociates.

An expression vector optionally includes one or more selection or marker sequences, which typically encode an enzyme capable of inactivating a compound in the growth medium. The inclusion of a marker sequence can, for example, render the host cell resistant to an antibiotic, or it can confer a compound-specific metabolic advantage on the host cell. Markers such as green fluorescent protein can also be used to monitor growth or toxicity in host cells in which it is expressed. Cells can be transformed with the expression vector using any convenient method known in the art, including chemical transformation, e.g., whereby cells are made competent by treatment with reagents such as CaCl₂); electroporation and other electrical techniques; microinjection, and the like. The vector may further include a tightly regulable expression control sequence operably linked to the nucleic acid sequence encoding the polypeptide, particularly a stabilized polypeptide, as described herein.

The term “introduced” in the context of inserting a nucleic acid sequence into a cell, means “transfection”, or “transformation” or “transduction” and includes reference to the incorporation of a nucleic acid sequence into a eukaryotic or prokaryotic cell wherein the nucleic acid molecule may be incorporated into the genome of a cell (e.g., chromosome, plasmid, plastid, or mitochondrial DNA), converted into an autonomous replicon, comprised in an episomal expression vector (see, e.g., Van Caenenbroeck et al., Eur. J. Biochem. 267:5665, 2000), or transiently expressed (e.g., transfected mRNA).

Host cell,” as used herein, refers broadly to a cell that contains an expression vector and supports the replication or expression of the expression vector. Host cells may be prokaryotic cells such as E. coli, or eukaryotic cells such as yeast, insect (e.g., SF9), amphibian, or mammalian cells such as CHO, HeLa, HEK-293, e.g., cultured cells, explants, and cells in vivo. The term “cell” is also used to include various cells derived from animals, individuals, subcultured cells, primary cultured cells, cell lines, recombinant cells, yeasts, microbes and the like.

The term “site” to which a peptide binds, i.e., the “site” that is recognized by a peptide is used to mean a consecutive or intermittent partial amino acid sequence or partial conformation on a target molecule to be bound or recognized by a peptide. In the present disclosure such a site can be referred to as an epitope or a binding site on the target molecule. In an embodiment, the site comprises the ligand binding domain of a steroid hormone receptor, such as, for example, an androgen receptor (AR) or an estrogen receptor (ER).

“Isolated,” as used herein, refers broadly to material removed from its original environment in which it naturally occurs, and thus is altered by the hand of man from its natural environment. Isolated material may be, for example, exogenous nucleic acid included in a vector system, exogenous nucleic acid contained within a host cell, or any material which has been removed from its original environment and thus altered by the hand of man (e.g., “isolated peptide”).

As used herein, the term “recombinant” refers to a cell, microorganism, nucleic acid molecule, or vector that has been modified by introduction of an exogenous nucleic acid molecule through human intervention, or refers to a cell or microorganism that has been altered such that expression of an endogenous nucleic acid molecule or gene is controlled, deregulated or constitutive, where such alterations or modifications are introduced or induced through human intervention (e.g., by genetic engineering). Genetic alterations may include, for example, modifications introducing nucleic acid molecules (which may include an expression control element, such as a promoter) encoding one or more proteins or enzymes, or other nucleic acid molecule additions, deletions, substitutions, or other functional disruption of or addition to a cell's genetic material. Exemplary modifications include those in coding regions or functional fragments thereof of heterologous or homologous polypeptides from a reference or parent molecule. A cell, microorganism, nucleic acid molecule, or vector that has been modified by introduction of an exogenous nucleic acid molecule may be referred to as “recombinant” or “non-naturally occurring” or “genetically engineered” or “transformed” or “transgenic”.

The terms “identical” or “percent identity,” in the context of two or more polypeptide or nucleic acid molecule sequences, means two or more sequences or subsequences that are the same or have a specified percentage of amino acid residues or nucleotides that are the same over a specified region (e.g., 60%, 65%, 70%, 75%, 80%, 85%, 90%, 91%, 92%, 93%, 94%, 95%, 96%, 97%, 98%, 99%, or 100% identity), when compared and aligned for maximum correspondence over a comparison window, or designated region, as measured using methods known in the art, such as a sequence comparison algorithm, by manual alignment, or by visual inspection. For example, preferred algorithms suitable for determining percent sequence identity and sequence similarity are the BLAST and BLAST 2.0 algorithms, which are described in Altschul et al. (Nuc'l. Acids Res. 25:3389, 1977) and Altschul et al. (J. Mol. Biol. 215:403, 1990), respectively.

The techniques and procedures are generally performed according to conventional methods well known in the art and as described in various general and more specific references that are cited and discussed throughout the present specification. See, e.g., Sambrook, et al. (2001) Molec. Cloning: Lab. Manual, 3^{rd} Ed, Cold Spring Harbor Laboratory Press. Standard techniques may be used for recombinant DNA, oligonucleotide synthesis, tissue culture, and transformation (e.g., electroporation, lipofection). Enzymatic reactions and purification techniques may be performed according to manufacturer's specifications or as commonly accomplished in the art or as described herein. The nomenclatures utilized in connection with, and the laboratory procedures and techniques of, analytical chemistry, synthetic organic chemistry, and medicinal and pharmaceutical chemistry described herein are those well-known and commonly used in the art. Standard techniques may be used for chemical syntheses, chemical analyses, pharmaceutical preparation, formulation and delivery, and treatment of patients.

“Treating” or “treatment,” as used herein, includes treatment of the disease or condition of interest in a mammal, preferably a human, having the disease or condition of interest, and includes: (a) inhibiting the disease or condition in a subject, i.e., arresting the disease or condition's development; (b) relieving (or ameliorating) the disease or condition, i.e., causing regression of the disease or condition; (d) relieving (or ameliorating) the symptoms resulting from the disease or condition, e.g., without addressing the underlying disease or condition; (e) preventing metastasis of the disease or condition; and/or (f) increasing or improving overall survival of the subject.

The term “therapeutically effective amount” or “an effective amount” refers to an amount of the drug or agent or a compound that may reduce the number of cancer cells; reduce the tumor size; inhibit (i.e., slow to some extent and particularly stop) cancer cell infiltration into peripheral organs; inhibit (i.e., slow to some extent and particularly stop) tumor metastasis; inhibit, to some extent, tumor growth; and/or relieve to some extent one or more of the symptoms associated with the disorder. To the extent the drug may prevent growth and/or kill existing cancer cells, it may be cytostatic and/or cytotoxic. For cancer therapy, efficacy in vivo can, for example, be measured by assessing the duration of survival, time to disease progression (TTP), the response rates (RR), duration of response, and/or quality of life.

Administration of the therapeutic compounds or agents to a patient or subject will follow general protocols for the administration of such compounds, taking into account the toxicity, if any, of the therapy. It is expected that the treatment cycle will be repeated as necessary. It also is contemplated that various standard therapies, as well as surgical intervention, may be applied in combination with the described therapy. The therapeutic agents disclosed herein may be combined with other therapeutic agents and/or modalities, such as surgery, radiation, immunotherapy, gene therapy, or a combination thereof.

As will be understood by those of ordinary skill in the art, the appropriate doses of the therapeutic agents/compositions disclosed herein will be approximately those already employed in clinical therapies wherein the therapeutic agent is administered alone or in combination with other agents or treatment modalities. Variation in dosage will likely occur depending on the condition being treated.

The instant description provides peptide/peptide memetic and other compositions for inhibiting the expression and activation of steroid hormone driven cancers. The steroid hormone receptors are members of the nuclear hormone receptor family. Nuclear hormone protein co-factors play a crucial role in regulating the biological functions of the androgen and estrogen receptors. They modulate the effects of nuclear hormone-driven gene activation. Alterations in co-factor recruitment can result in changes in gene expression. The synthetic PRAC1 peptides and peptide memetics disclosed herein bind to and inhibit the interaction of androgen and estrogen receptor with one or more of their co-factors and specific ligands. These interactions are expected to suppress or inhibit a steroid hormone driven cancer, such as, for example, prostate, uterine, ovarian, and/or breast cancer growth in vitro and in vivo.

A method for treating a steroid hormone driven cancer described herein may be performed alone or in conjunction with an additional therapy or therapeutic modality, such as chemotherapy, radiation therapy, surgery, hormone therapy, gene therapy, immunotherapy, chemoimmunotherapy, cryotherapy, ultrasound therapy, liver transplantation, local ablative therapy, radiofrequency ablation therapy, photodynamic therapy, and the like. The additional therapeutic modality may be administered before, after, sequentially, concurrently, or simultaneously with a method disclosed herein.

For prostate cancer the combined therapies can include therapies that target the androgen receptor (AR) signaling axis including, but not limited to, leuprorelin (leuprolide), goserelin, triptorelin, histrelin, buserelin, and degarelix, flutamide, nilutamide, enzalutamide, apalutamide, darolutamide, galeterone, abiraterone acetate, seviteronel, and the like. Therapies targeting Poly (ADP-ribose) polymerase (PARP) including but not limited to, olaparib, rucaparib, niraparib, talazoparib, and the like. Therapies directed against cell surface targets including, but not limited to, IMMU-132 (sacituzumab-govitecan), trastuzumab-DXd, a PSMA specific antibody conjugated to monomethyl auristatin E (PSMA-MMAE), Dato-DXd, and the like. Therapies involving radioligands including but not limited to ¹⁷⁷Lu-PSMA-617, radium-223, and the like. Therapies involving chemotherapeutic drugs, including but not limited to docetaxel, cabazitaxel, mitoxantrone, estramustine, carboplatin, and the like. Therapies that involve external beam radiation and/or brachytherapy, and therapies that involve surgery. Therapies that involve immunotherapy, including but not limited to Sipuleucel-T, pembrolizumab, ipilimumab, nivolumab, atezolizumab, durvalumab, avelumab, and the like.

For breast, ovarian, and/or uterine cancers the combined therapies that can include, for example, hormonal therapies, including but not limited to tamoxifen, toremifene, fulvestrant anastrozole, exemestane, letrozole, goserelin, leuprolide, and the like. Therapies involving chemotherapies, including but not limited to, cyclophosphamide, methotrexate, 5-fluorouracil, doxorubicin, paclitaxel, docetaxel, epirubicin, and the like. Targeted therapies, including but not limited to abemaciclib, alpelisib, palbociclib, ribociclib, everolimus, olaparib, rucaparib, talazoparib, lapatinib, neratinib, bevacizumab, and the like. Therapies directed against cell surface antigen, including but not limited to, Trastuzumab, Pertuzumab, Ado-trastuzumab emtansine, trastuzumab-deruxtecan, IMMU-132 (sacituzumab-govitecan), trastuzumab-DXd, Dato-DXd, and the like. Therapies involving external beam radiation, brachytherapy, and/or surgery. Immunotherapies, including but not limited to pembrolizumab, ipilimumab, nivolumab, atezolizumab, durvalumab, avelumab, and the like.

The terms “drug,” “therapeutic agent,” and “beneficial agent” are used interchangeably to refer to any therapeutically active substance that is delivered to a subject to produce a desired beneficial effect. In one embodiment of the present disclosure, the drug is an isolated peptide/peptidomimetic as described herein. In another embodiment of the present disclosure, the drug is a small molecule. The devices and methods of the present disclosure are well suited for the delivery of proteins, peptides, small molecules, and combinations thereof.

Prostate cancer (PCa) is an androgen receptor dependent disease. Treatments are often aimed at the androgen receptor, ligand binding to the receptor, or androgen mediated intracellular signaling pathways. PCa has been shown to circumvent these treatment pathways (resistance, resistance to treatment, treatment failure) by processes of selectivity and treatment pressures, to mutate the key proteins involved in the proliferation and “health” of the tumor. Mutations or genetic alterations may result in gain or loss of function, increased or decreased ligand binding, increased or decreased gene expression (changes or selectivity on gene expression of the androgen receptor itself or a gene product that is involved in steroid receptor activity), increase or decrease of steroid receptor DNA binding, receptor constitutive activity or loss of ligand responsivity, changes to the ability of the receptor to dimerize, changes to ligand binding sites on the effector proteins—e.g., cofactors become enhancers or inhibitors, antagonists become agonists, ligand promiscuity (e.g., progesterone, hydrocortisone, estrogen, and Cortisol under normal conditions do not bind the androgen receptor and ligand binding mutations within the androgen receptor may allow binding and activation by these other physiological relevant steroids).

Once prostate cancer is diagnosed and staged, clinical management options include expectant, regular, or interval management or surveillance, surgery, radiation therapy, cryosurgery, hormone therapy, chemotherapy, immunotherapy, and vaccine treatment. Often the inclusion of age and expected life span and other concomitant health conditions are considered along with the stage and grade of the tumor in the treatment options. Since prostate cancer is an androgen-dependent disease, hormone therapy or androgen deprivation therapy (ADT) or androgen suppression therapy has the overall goal of reducing the levels of androgens in the body or to prevent them from reaching the prostate cancer cells (chemical castration). Hormone therapy includes LHRH agonists (Lupron, eligard, goserelin, triptorelin, histrelin); LHRH antagonists (firmagon). Hormone therapy may be used in conjunction with surgical resection of the tumor, orchiectomy (surgical castration), or radiation therapy or radiopharmaceutical (Radium 223 Dichloride, Xofigo (Radium 223 Dichloride). Therapy aimed at reducing the production of androgens include abiraterone (a CYP17 inhibitor). Anti-androgen therapy is aimed at inhibiting the androgen receptor and examples are flutamide, bicalutamide, nilutamide, ARN-509 and enzalutamide. Anti-androgen treatment may be combined with orchiectomy or LHRH analogs as first-line hormone therapy. This is called combined androgen blockade (CAB). Other androgen suppressing drugs include estrogens and ketoconazole. Thus, targeting the androgen receptor signaling pathway has been a drug development staple and broadly includes CYP17 inhibitors or modulators, antiandrogens, chaperone inhibitors (targeting heat shock proteins, Hsp-27 inhibitor), androgen-receptor modulator (blocking transactivation domain of the receptor).

Vaccine treatment, currently Sipuleucel, is intended to boost the body's immune system to recognize the prostate tumor and lodge an anti-tumor immune response. This form of therapy is not “off the shelf as each vaccine is made from the unique white cells from each individual patient after exposing in a lab to prostate acid phosphatase (PAP). Another immunotherapy includes ipilimumab (a CTLA-4 antagonist). Castration resistant prostate cancer (CRPC) is the term used for those patients for which androgen deprivation or androgen suppression therapy is no longer effective at slowing the proliferation of the prostate tumor or the metastasis, and it is a stage of the disease that is associated with primary or acquired resistance to therapy and for which there are few therapeutic options.

Using an integrated epigenome and transcriptome screen, the inventors of the subject matter encompassed in the present disclosure have identified PRAC1 as a gene locus that undergoes CpG methylation mediated silencing in approximately 30% of treatment refractory prostate cancer. Mechanistically, PRAC1 is involved in mediating canonical androgen receptor signaling. Loss of PRAC1 results in transcriptional reprogramming and resistance to hormonal therapy. These observations constitute a completely novel resistance mechanism and nominate/identify PRAC1 as a potential new target for drug development in advanced PC.

PRAC1 nucleotide sequence
(SEQ ID NO: 1)
GTCCTTCCTCTCCTAGCCTAAGGCGTGCAAACAGAGCGCCACTGGGAGGC
TGAAACCTTTAGGCCGATGCTTGCTTGCAAGGTCAGGCAAGCTGGATTCT
GGTCCCCACCTTTGCAGAGAGAACAGCGATGTTGTGCGCCCATTTCTCAG
ATCAAGGACCGGCCCATCTTACTACCTCCAAGAGTGCTTTTCTCTCTAAT
AAGAAAACATCTACTTTGAAACATCTACTGGGCGAGACCAGGAGTGATGG
CTCAGCCTGTAATTCTGGAATTTCGGGAGGCCGAGGCAGGAAGATTCCTT
GAGCACAGGAGTTCCAGACCAGCCTGGGCAATGTAGCAAGACGCTGTCTC
TATTTATACAATAAAATTTTTTTAAAAAAGGAAAAAAAAAAAAAAAAAAA
AAA

The present disclosure provides PRAC1 as a novel targetable mechanism of resistance to hormonal therapy in prostate cancer. The inventors have demonstrated that loss of PRAC1 by CpG methylation is a biomarker for predicting response to hormonal therapy and/or overall disease-free survival. Specifically, the inventors show that that PRAC1 expression is higher in responders compared to non-responders of hormonal therapy in Prostate cancer patients. Furthermore, patients with low PRAC1 expression show earlier disease progression when treated with androgen receptor signaling inhibitors, such as, for example, enzalutamide. Moreover, the inventors demonstrate that PRAC1 binds to the ligand binding domain of androgen receptor (AR) to result in major changes in gene transcription involving numerous AR regulated genes. Further, the data disclosed herein demonstrate that the reconstitution of PRAC1 expression in prostate cancer resistant to androgen deprivation therapy results in restoration of sensitivity to ADT or androgen receptor signaling inhibitors, such as, for example, enzalutamide, and suppresses prostate cancer growth in vitro and in vivo.

In some embodiments, also provided herein are PRAC1 peptides/peptidomimetics, derivatives and precursors thereof. In some embodiments, the PRAC1 peptides/peptidomimetics of the present disclosure comprise one of more substitutions, deletions, or modifications relative to the amino acid sequence of the wild-type PRAC1 and which may have the property of an improved biological activity in vivo, improved, and prolonged binding to androgen receptor ligand binding domain and/or improved chemical stability, e.g., as assessed in in vitro stability assays. These modifications include appendage of cell-permeable, hydrophilic sequences, cyclization, and introduction of peptide “staples.” The inventors have demonstrated that PRAC1 binds to the ligand binding domain of the AR and ER. Thus, relative to the amino acid sequence of the wild-type peptide, any amino acid which is not essential to the androgen receptor or estrogen receptor binding activity of the PRAC1 peptide may be replaced with any other amino acids, while amino acids which are essential to the androgen receptor binding activity of the PRAC1 peptide may be replaced only with amino acid analogs which do not substantially decrease said activity.

More particularly, preferred PRAC1 peptides/peptidomimetics or derivatives thereof of the present disclosure may comprise deletions at one or more of positions of the wild-type PRAC-1 sequence, optionally in combination with further substitutions and/or a deletion of one or more of amino acids and/or the addition of a N-terminal or C-terminal stabilizing peptide sequence and/or the addition of a N-terminal or C-terminal cell penetrating peptide.

As well as providing PRAC1 analogues or derivatives that may have improved chemical stability and/or biological activity, the present disclosure also relates to providing compounds, peptides, or peptidomimentics that have preferential growth suppressing activity in a malignant condition, for e.g., prostate, uterine, ovarian, and/or breast cancer, in particular by including deletion at the amino terminus of an isolated peptide comprising the wild-type PRAC1.

In some embodiments, the present disclosure provides an isolated peptide comprising the full-length wild-type PRAC1 protein. In some embodiments, the isolated peptide comprises a peptide encoded by ORF1 of PRAC1 gene. In some embodiments, the isolated peptide comprises amino acid residues capable of binding to the ligand binding domain of the androgen receptor (AR). In some embodiments, the isolated peptide comprises the c-termini amino acid residues of the PRAC1 protein encoded by ORF1 of PRAC1 gene.

ORF1 nucleotide sequence
(SEQ ID NO: 2)
ATGCTTGCTTGCAAGGTCAGGCAAGCTGGATTCTGGTCCCCACCTTTGC
AGAGAGAACAGCGATGTTGTGCGCCCATTTCTCAGATCAAGGACCGGC
CCATCTTACTACCTCCAAGAGTGCTTTTCTCTCTAATAAGAAAACATCT
ACTTTGA
ORF1 full length amino acid sequence
(SEQ ID NO: 3)
MLACKVRQAGFWSPPLQREQRCCAPISQIKDRPILLPPRVLFSLIRKHLL

In some embodiments, the isolated peptide comprises 20, 19, 18, 17, 16, 15, 14, 13, 12, 11, 10, 9, 8, 7, 6, or 5 carboxy-terminal amino acid residues of the PRAC1 protein encoded by ORF1 of PRAC1 gene. In some embodiments, the isolated peptide comprises 14 or 10 carboxy-terminal amino acid residues of the PRAC1 protein encoded by ORF1 of PRAC1 gene.

(SEQ ID NO: 9)
PPRVLFSLIRKHLL
(SEQ ID NO: 34)
LFSLIRKHLL

In some embodiments, the isolated peptide further comprises a basic stretch of amino acid residues added at the carboxy-terminus. For example, in certain embodiments presented herein the isolated peptide can comprise:

(SEQ ID NO: 8)
PPRVLFSLIRKHLLRKKRRQRRRG
(SEQ ID NO: 10)
LFSLIRKHLLRKKRRQ

In some embodiments, the isolated peptide further comprises a peptide “staple” to reinforce the peptide helical structure. For example, in certain embodiments specifically disclosed herein the isolated peptide can comprise:
PPXVLFSLIXKHLLRKKRRQRRRG, wherein X³ is R-2-(7′octanyl) alanine, and X¹⁰ is S-2-(4′-pentenyl) alanine (SEQ ID NO:38)
PPXVLFSLIXKHLL, wherein X³ is R-2-(7′octanyl) alanine, and X¹⁰ is S-2-(4′-pentenyl) alanine (SEQ ID NO:39)

In some embodiments, the isolated peptide can further comprise 5, 6, 7, 8, 9, 10, or more additional flanking non-structural amino acids adjacent to the AR interacting helix that are linked via head-to-tail amide cyclization. In particular examples disclosed herein the isolated peptide can comprise:

(SEQ ID NO: 40)
XXXXXSLIRKHL
(SEQ ID NO: 41)
XXXXXSLIRKHLL
(SEQ ID NO: 42)
SLIRKHLXXXXX
(SEQ ID NO: 43)
SLIRKHLLXXXXX
(SEQ ID NO: 44)
XXXXXXSLIRKHL
(SEQ ID NO: 45)
XXXXXXSLIRKHLL
(SEQ ID NO: 46)
SLIRKHLXXXXXX
(SEQ ID NO: 47)
SLIRKHLLXXXXXX
(SEQ ID NO: 48)
XXXXXXXSLIRKHL
(SEQ ID NO: 49)
XXXXXXXSLIRKHLL
(SEQ ID NO: 50)
SLIRKHLXXXXXXX
(SEQ ID NO: 51)
SLIRKHLLXXXXXXX
(SEQ ID NO: 52)
XXXXXXXXSLIRKHL
(SEQ ID NO: 53)
XXXXXXXXSLIRKHLL
(SEQ ID NO: 54)
SLIRKHLXXXXXXXX
(SEQ ID NO: 55)
SLIRKHLLXXXXXXXX
(SEQ ID NO: 56)
XXXXXXXXXSLIRKHL
(SEQ ID NO: 57)
XXXXXXXXXSLIRKHLL
(SEQ ID NO: 58)
SLIRKHLXXXXXXXXX
(SEQ ID NO: 59)
SLIRKHLLXXXXXXXXX
(SEQ ID NO: 60)
SGPLGSLIRKHL
(SEQ ID NO: 61)
SLIRKHLSGPLG
(SEQ ID NO: 62)
SGPLGSLIRKHLL
(SEQ ID NO: 63)
SLIRKHLLSGPLG
(SEQ ID NO: 64)
XXXXXXLFSLIRKHLL
(SEQ ID NO: 65)
XXXXXXXLFSLIRKHLL
(SEQ ID NO: 66)
XXXXXXXXLFSLIRKHLL
(SEQ ID NO: 67)
XXXXXXXXXLFSLIRKHLL
(SEQ ID NO: 68)
LFSLIRKHLLXXXXXX
(SEQ ID NO: 69)
LFSLIRKHLLXXXXXXX
(SEQ ID NO: 70)
LFSLIRKHLLXXXXXXXX
(SEQ ID NO: 71)
LFSLIRKHLLXXXXXXXXX
(SEQ ID NO: 72)
SGPLGLFSLIRKHLL
(SEQ ID NO: 73)
LFSLIRKHLLSGPLG

wherein X when present comprises any non-helical sequence of flexible, cell-permeable, and ring-forming amino acids, i.e., G, S, P, D, N, R, A, Q, C, H, Y, W, T, F, or K.

The isolated peptide of the present disclosure, or a derivative thereof may comprise a signal sequence. A signal sequence present at or added to the N terminus of a certain polypeptide, a derivative, or a precursor thereof is useful for delivering the polypeptide to a specific compartment of a cell. Many signal sequences are known to a person skilled in the art, and the signal sequence can be selected according to host cells. The peptide of the present disclosure, a precursor, or a derivative thereof may comprise a cell-penetrating sequence. A cell-penetrating amino acid sequence at or added to the C-terminus of a certain polypeptide a derivative, or a precursor thereof is useful for delivering the polypeptide to the cell. Many cell-penetrating sequences are known to a person skilled in the art, and the signal sequence can be selected accordingly. In a specific example disclosed herein the cell-penetrating peptide comprises part of the TAT proteins and comprises the amino acid residue sequence:

(SEQ ID NO: 7)
RKKRRQRRRG

The isolated peptide of the present disclosure can be tagged in advance and thereby purified by affinity chromatography. The peptide of the present disclosure can comprise, for example, biotin, Strep Tag®, Strep tag II®, oligohistidine such as His6, polyhistidine, an immunoglobulin domain, a maltose-binding protein, glutathione-S-transferase (GST), a calmodulin-binding peptide (CBP), a hapten such as digoxigenin or dinitrophenol, an epitope tag such as FLAG®, myc tag, or HA tag (hereinafter, collectively referred to as an “affinity tag”) at its C-terminus. The tagged form can also be included in some aspects of the conjugate of the present disclosure. The conjugate of the present disclosure may be a peptide (polypeptide) as a whole.

The isolated peptide of the present disclosure can comprise a moiety for labeling. Specifically, the peptide of the present disclosure can be conjugated to a label moiety such as an enzymatic label, a radiolabel, a colored label, a fluorescent label, a coloring label, a luminescent label, a hapten, digoxigenin, biotin, a metal complex, a metal, or colloidal gold. The form comprising the moiety for labeling can also be included in some aspects of the peptides of the present disclosure.

The isolated peptide of the present disclosure can comprise any of the natural amino acids and non-natural amino acids in its peptide moiety and can comprise an L-amino acid and a D-amino acid as a natural amino acid. The amino acid sequence of the peptide of the present disclosure can comprise any of the natural amino acids and non-natural amino acids and can comprise an L-amino acid and a D-amino acid as a natural amino acid.

In an embodiment, the present disclosure provides an isolated peptide comprising a polypeptide at least about 95%, 90%, 80%, 70%, 60%, 50%, 40%, 30%, 20%, 10% identical to the amino acid sequence set forth in SEQ ID NO:3, SEQ ID NO:9, or SEQ ID NO: 34. In some embodiments, the present disclosure provides a composition comprising: an isolated peptide comprising the amino acid sequence at least about 90% identical to the amino acid sequence set forth in SEQ ID NO:3, SEQ ID NO:9, or SEQ ID NO: 34. In some embodiments, the composition further comprises a pharmaceutically acceptable carrier.

The present disclosure also provides a polynucleotide encoding the isolated peptide comprising the amino acid sequence at least about 95%, 90%, 80%, 70%, 60%, 50%, 40%, 30%, 20%, 10% identical to the amino acid sequence set forth in SEQ ID NO:3, SEQ ID NO:9, or SEQ ID NO:34. In some embodiments, provided herein is a polynucleotide encoding the isolated peptide comprising the amino acid sequence at least about 90% identical to the amino acid sequence set forth in SEQ ID NO:3, SEQ ID NO:9, or SEQ ID NO:34. In some embodiments, provided herein is a polynucleotide set forth in SEQ ID NO:2 or SEQ ID NO:6. In some embodiments, the polynucleotide encodes an isolated peptide consisting of the amino acid sequence of SEQ ID NO:3, SEQ ID NO:9, or SEQ ID NO:34.

To further increase PRAC1 peptide stability, affinity for the androgen receptor, and bioactivity, the isolated PRAC1 peptides/peptidomimetics disclosed herein may comprise at least one modification. In some embodiments, the carboxy-terminus of the isolated peptide of the disclosure is extended with a sequence of amino acid residues that enhances delivery to a cell, for e.g., amino acid residues encoding a cell penetrating peptide. In some embodiments, the carboxy-terminus of the peptides disclosed herein is extended with a sequence selected from (KKRR) 6 (SEQ ID NO:74), RKKRRQ (SEQ ID NO: 75), or RKKRRQRRRG (SEQ ID NO:7). In some embodiments, the at least one modification comprises addition of a basic stretch of amino acids to the C-terminus of the peptides disclosed herein.

In some embodiments, the modification comprises modification of at least two amino acid residues of the PRAC1 peptides/peptidomimetics disclosed herein. In some embodiments, the modification of the at least two amino acid residues of the PRAC1 peptide/peptidomimetic disclosed herein comprises cross-linking and or stapling to the at least two amino acid residues. Techniques and methodologies utilized in selecting amino acid residues for the introduction of a peptide staple and cyclization of the minimal bioactive sequence include but are not limited to crystal structure of the AR or ER LBD bound to PRAC1 derivatives, in vivo alanine scan, and structural modeling using AlphaFold. The criteria for selection include amino acid residues that are outside of the PRAC1 bioactive core and that do not make intra- or intermolecular interactions with androgen receptor (AR) and/or the estrogen receptor (ER), and more specifically with the ligand binding domain of the AR and/or ER, based on experimental data (crystal structures). Thus, identified key amino acid residues of the PRAC1 peptide/peptidomimetic may be leveraged for stapling or cyclization.

In an embodiment, the present disclosure provides a composition comprising an isolated peptide/peptidomimetic comprising an amino acid sequence at least 60%, 70%, 80%, 85%, 90% or 95% identical to the amino acid sequence as set forth in SEQ ID NO:8 and/or SEQ ID NO:9. A (Stapled peptide #1) or B (Stapled peptide #2), wherein at least two non-native amino acids substitute PRAC1 derivative residues not involved in the PRAC1-AR interaction, permitting cross-linking to reinforce the PRAC1 alpha-helix, wherein the cross-linker comprises R-2-(7′-octenyl) alanine (also referred to as “R8”) and S-2-(4′-pentenyl) alanine (also referred to as “S5”).
PPX³VLFSLIX¹⁰KHLLRKKRRQRRRG, wherein X³ is R-2-(7′-octanyl) alanine, and X¹⁰ is S-2-(4′-pentenyl) alanine SEQ ID NO:38
PPX³VLFSLIX¹⁰KHLL, wherein X³ is R-2-(7′-octanyl) alanine, and X¹⁰ is S-2-(4′-pentenyl) alanine SEQ ID NO:39

Macrocyclization between head, tail, or sidechains is a frequently employed strategy to enhance peptide and peptidomimetic stability, selectivity, and affinity. Head to tail amide cyclization will minimize peptide size, bypassing the need for an additional solubility motif.

In an embodiment, the present disclosure provides a PRAC1 peptide/peptidomimetic capable of binding to the ligand binding domain of the androgen receptor. In an embodiment, provided herein is a composition comprising a peptidomimetic macrocycle comprising an amino acid sequence at least about 50%, 60%, 70%, 80%, 85%, 90% or 95% identical to the amino acid sequence as set forth in SEQ ID NO: 60 (Cyclic peptide #1), SEQ ID NO:62 (Cyclic Peptide #2), SEQ ID NO:63 (Cyclic Peptide No: 3), SEQ ID NO:64 (Cyclic Peptide No: 4), SEQ ID NO:72 (Cyclic Peptide No: 13), or SEQ ID NO:73 (Cyclic Peptide NO: 14), wherein the linear amino acid sequence presented is chemically coupled and cyclized through N-terminus to C-terminus cyclization. In an embodiment, the present disclosure provides a composition comprising an isolated peptide/peptidomimetic comprising an amino acid sequence set forth in SEQ ID NOs: 40 to 59, and 64 to 71 wherein X when present comprises any non-helical sequence of flexible, cell-permeable, and ring-forming amino acid residues, i.e., G, S, P, D, N, R, A Q, C, H, Y, W, T, F, and/or K.

In some embodiments, the present disclosure provides a method for predicting response to a therapy targeting the androgen receptor (AR) signaling axis, such as, for example, androgen deprivation therapy (ADT), for a subject suffering from prostate cancer. In some embodiments, the method comprises: obtaining one or more biological samples from the subject; measuring the expression level of at least one biomarker gene in the one or more biological samples; comparing the expression levels of the at least one biomarker in the one or more biological sample obtained from the subject with an expression level of the at least one biomarker in a reference/control sample; and identifying the subject as responsive or non-responsive to the therapy targeting the AR signaling axis when the at least one biomarker gene is differentially expressed in the biological sample obtained from the subject relative to the expression levels of the biomarker in the reference or control sample. In some embodiments, the at least one biomarker gene comprises Prostate Cancer Susceptibility Candidate 1 (PRAC1).

In some embodiments, the present disclosure also provides a method for a method of selecting treatment for prostate cancer in a subject. The method comprising administering to the subject a pharmaceutical composition comprising a peptide and/or peptide mimetic disclosed herein alone or together with a therapy targeting the AR signaling axis, such as, for example, androgen deprivation therapy (ADT) if the subject has been identified as responsive to ADT, or administering to the subject an alternative treatment other than ADT if the subject has been identified as non-responsive to ADT. In some embodiments, the subject is identified as responsive or non-responsive to ADT by a method comprising: obtaining one or more biological samples from the subject; measuring the expression level of at least one biomarker gene comprising PRAC1 in the one or more biological samples; comparing the expression levels of the at least one biomarker gene comprising PRAC1 in the one or more biological sample obtained from the subject with an expression level of PRAC1 in a reference/control sample. In an embodiment, a differential expression of PRAC1 in the biological sample obtained from the subject relative to the expression levels of PRAC1 in the reference or control sample identifies the subject as responsive or non-responsive to ADT.

Also provided herein is a method of predicting earlier disease progression in a subject suffering from prostate cancer. In some embodiments, the method comprises obtaining one or more biological samples from the subject; measuring the expression level of at least one biomarker gene comprising PRAC1 in the one or more biological samples; comparing the expression levels of the at least one biomarker gene comprising PRAC1 in the one or more biological sample obtained from the subject with an expression level of PRAC1 in a reference/control sample. In an embodiment, a lower expression of PRAC1 in the biological sample obtained from the subject relative to the expression levels of PRAC1 in the reference or control sample predicts earlier disease progression in the subject.

In an embodiment, measuring the expression level comprises measuring/quantifying a transcript of PRAC1 and/or a protein encoded by PRAC1 gene. In a related embodiment, measuring the expression level further comprises determining the methylation status of PRAC1 gene locus in the biological sample obtained from the subject.

In some embodiments, the differential expression comprises a lower expression of PRAC1 (PRAC1^{low}) in the one or more biological sample obtained from the subject relative to the reference/control sample. In some embodiments, the lower expression of PRAC1 in the biological sample obtained from the subject relative to the reference/control identifies the subject as non-responsive or resistant to a therapy targeting the androgen receptor signaling axis.

In some embodiments, the differential expression comprises a higher or a similar expression of PRAC1 (PRAC1^{high}) or a similar expression of PRAC1 in the one or more biological sample obtained from the subject relative to the reference/control sample. In some embodiments, the higher or similar expression of PRAC1 in the biological sample obtained from the subject relative to the reference/control identifies the subject as responsive or sensitive to a therapy targeting the AR signaling axis.

For comparison of the levels of biomarkers disclosed herein, a number of references or controls would be considered appropriate. For example, a biomarker level could be compared with the level of the biomarker in tissue known to be non-cancerous prostate tissue. A subject's own tissue could be used as a reference for comparison, or a population-derived value may be obtained. Local reference standards can be established if it is found that epidemiological variation exists among populations studied. Standards established within a group having a common demographic may also be established based on, for example, age, sex, smoking status, and other potentially influencing factors.

Thus, in some embodiments, the reference sample is a biological sample obtained from a healthy subject, wherein the healthy subject is a subject not suffering from Prostate cancer or diagnosed with Prostate cancer. In some embodiments, the reference sample is a biological sample corresponding to the biological sample obtained from the subject. The reference/control expression levels of the one or more biomarkers disclosed herein may be determined from a level of the one or more biomarkers in known non-cancerous prostate tissue or known prostate cancer tissue. The reference/control expression levels of the one or more biomarkers disclosed herein can also be determined from a level of the one or more biomarkers in the subject's own noncancerous tissue.

Statistical methods can be used to define the range of values for a reference/control. A range could be values within one standard deviation of the mean, and preferably values within two standard deviations of the mean. A level of biomarker within such a range or outside of the defined range as assessed, measured, or determined in a biological sample obtained from a subject may indicate that the subject is responsive or non-responsive to androgen deprivation therapy, respectively. An alternate reference for comparison of the biomarker level could be determined by establishing the level of biomarker in tissue known to be responsive to androgen deprivation therapy. Statistical methods can be used to define the range of values for the alternate reference. A range could be values within one standard deviation of the mean, and preferably values within two standard deviations of the mean.

Biomarker levels may be compared with a threshold level beyond which a positive indication is determined. For example, if a particular biomarker is shown to exhibit an increase (or decrease) relative to a reference/control, a threshold level of change, for example, a 5%, 10%, 15%, 20%, 25%, 30%, 35%, 40%, 45%, 50%, 75%, 100%, 125%, 150%, 200%, 300%, 400% or 500% increase, or a 5%, 10%, 15%, 20%, 25%, 30%, 35%, 40%, 45%, 50%, 75% or 100% decrease, relative to a reference/control may be set or pre-determined to allow a comparison from which a positive or negative indication can be derived.

Those of skill in the art are familiar with differentiating between significant up-regulation or down-regulation of expression of a biomarker relative to a reference or background expression of a biomarker. When a biomarker is up regulated or overexpressed as an indication of a positive result, this can be easily distinguished from background (low-level values). Background expression levels may be used to form a “cut-off′ above which increased staining will be scored as a significant positive expression. Positive expression may be represented by a high level of antigen in tissue, or by a high proportion of cells from within a tissue that give a positive signal. When down-regulation of a biomarker is indicative of a positive result, those of skill in the art are also familiar with differentiating between significantly reduced expression of a biomarker, and background expression of a biomarker at a higher level. Indeed, background expression levels in such instances may be used to determine a “cut-off′ below which decreased staining will be scored as lower expression. Lower expression may be represented by low levels of antigens in tissues, or alternatively, by a low proportion of cells from within a tissue that each give a low signal.

A biomarker can be determined to be statistically different from a reference standard or background if the mean or median expression level of the biomarker in a group forming a reference versus a group representing a positive diagnosis of the disease condition is to be statistically significant, as was the case in the present disclosure. Common tests for statistical significance include, among others, t-test, ANOVA₁ Kruskal-Wallis, Wilcoxon, Mann-Whitney and odds ratio. Biological samples (of unknown status) can be compared with data from the reference group (negative control), and/or compared with data obtained from a positive control group known to have cancer.

As used herein, the phrases “indicative of” or “diagnosing” or “identifies”, when referring to levels of a biomarker or an expression pattern of a biomarker which is diagnostic or confirmatory of disease such that the biomarker levels or expression pattern is found significantly more often in subjects with a disease than in subjects without the disease (as determined using routine statistical methods setting confidence levels at a typical minimum, such as at 95%). Preferably, an expression pattern which is indicative of disease is found in at least 70%, at least 75%, at least 80%, at least 85%, at least 90%, at least 95% or more in subjects who have the disease, and is found in less than 10%, less than 8%, less than 5%, less than 2.5%, or less than 1% of patients who do not have the disease. The phrase may also indicate an expression pattern which is diagnostic of disease such that the expression pattern more properly categorizes expression patterns of individuals/subjects with disease as compared with control expression patterns of individuals/subjects without disease using statistical algorithms for class prediction. Such comparisons would readily be understood by a person skilled in the art, and could be implemented using computerized means, such as for example commercially available programs available from Silicon Genetics (e.g., GeneSpring™).

Expression levels or levels in a subject can be measured in cells of a biological sample obtained from the subject by methods known to those skilled in the art. For example, a tissue sample can be removed from a subject by conventional biopsy techniques. In another example, a body fluid sample, such as a lymph, blood or serum sample, or an exudate fluid sample such as a cancerous organ exudate may be used as the sample. A blood sample can be removed from the subject and white blood cells can be isolated for DNA extraction by standard techniques. The fluid or tissue sample obtained from the subject can be done prior to the initiation of radiotherapy, chemotherapy, or other therapeutic treatment. A corresponding control tissue or blood sample can be obtained from unaffected or non-disease state tissues of the subject, from a normal (non-disease or non-cancerous) subject or population of normal subjects, or from cultured cells corresponding to the majority of cells in the subject's sample. The control tissue or blood sample is then processed along with the sample from the subject, so that the levels of expression in cells from the subject's sample can be compared to the corresponding expression levels from cells of the control sample.

The level of a gene product in a sample can be measured using any technique that is suitable for detecting RNA expression levels in a biological sample. Suitable techniques for determining RNA expression levels in cells from a biological sample are well known to those of skill in the art, including, for example, Northern blot analysis, RT-PCR, in situ hybridization, RNA-sequencing, and the like.

A variety of methodologies can be utilized to assess, quantify expression levels and/or expression patterns of the one or more biomarkers disclosed herein in fine needle aspiration biopsy specimens and/or other biological samples or tissues obtained from a subject. In some embodiments, assessing levels and/or expression levels can comprise assessing and/or measuring/quantifying levels of DNA, transcript, and/or protein or expression patterns for DNA encoding the one or more biomarker, RNA transcript, protein, and/or a combination thereof of the one or more biomarkers, DNA methylation detection and/or quantification of the one or more biomarkers disclosed herein in a biological sample obtained from a subject.

A number of proteomic techniques which can detect biomarkers are known in the art. Such techniques include, but are not limited to: Western blot analysis, enzyme linked immunosorbent assay (ELISA) or mass spectrometry, and the like.

Another suitable method is RIA (radioimmunoassay). An example of RIA is based on the competition between radiolabeled-polypeptides and unlabeled polypeptides for binding to a limited quantity of antibodies. Suitable radiolabels include, but are not limited to, I¹²⁵. In one embodiment, a fixed concentration of I¹²⁵-labeled polypeptide is incubated with a series of dilution of an antibody specific to the polypeptide. When the unlabeled polypeptide is added to the system, the amount of the I′-polypeptide that binds to the antibody is decreased. A standard curve can therefore be constructed to represent the amount of antibody-bound I′-polypeptide as a function of the concentration of the unlabeled polypeptide. From this standard curve, the concentration of the polypeptide in unknown samples can be determined. Various protocols for conducting RIA to measure the levels of polypeptides in cell samples are well known in the art.

Suitable antibodies include, but are not limited to, polyclonal antibodies, monoclonal antibodies, chimeric antibodies, humanized antibodies, single chain antibodies, Fab fragments, and fragments produced by a Fab expression library.

Antibodies can be labeled with one or more detectable moieties to allow for detection of antibody-antigen complexes. Detectable moieties can include compositions detectable by spectroscopic, enzymatic, photochemical, biochemical, bioelectronic, immunochemical, electrical, optical, or chemical means. The detectable moieties include, but are not limited to, radioisotopes, chemiluminescent compounds, labeled binding proteins, heavy metal atoms, spectroscopic markers such as fluorescent markers and dyes, magnetic labels, linked enzymes, mass spectrometry tags, spin labels, electron transfer donors and acceptors, and the like. Different antibodies can be evaluated, and optimization procedures carried out (for example antibody dilutions and antigen retrieval methodologies), scoring systems and cut-off points of significance utilized, and various methods of data analysis can be employed according to the invention.

Determination of a biomarker can also be undertaken at the DNA level. Detection of DNA can be achieved using methods known in the art, such as DNA/RNA sequencing, for example, Next Generation Sequencing (NGS) methods, Southern blot analysis, microarrays or other techniques known in the art.

Methods for measuring site specific methylation changes include, for example, sodium bisulfite sequencing, methylation-specific real time PCR, methyl-binding domain (MBD2) sequencing, methylated DNA immunoprecipitation (MEDIP or MeDIP) and enzymatic methyl-seq (EM-seq).

In accordance with one aspect, the differential expression patterns of the one or more biomarkers disclosed herein can be determined by measuring the levels of RNA transcripts of these genes, or genes whose expression is modulated by these genes, in the biological sample obtained from the subject. Suitable methods for this purpose include, but are not limited to, RNA-sequencing, RT-PCR, Next Generation Sequencing, Northern Blot, in situ hybridization, Southern Blot, slot-blotting, nuclease protection assay, and oligonucleotide arrays.

Detection of RNA can be achieved using methods known in the art, such as northern blot analysis, microarrays, exon arrays, RNA-sequencing, or other transcriptome-based techniques well known in the art.

In certain aspects, RNA isolated from the biological sample obtained from the subject can be amplified to cDNA or cRNA before detection and/or quantitation. The isolated RNA can be either total RNA or mRNA. The RNA amplification can be specific or non-specific. Suitable amplification methods include, but are not limited to, reverse transcriptase PCR, isothermal amplification, ligase chain reaction, and Qbeta replicase. The amplified nucleic acid products can be detected and/or quantitated through hybridization to labeled probes. In some embodiments, detection may involve fluorescence resonance energy transfer (FRET) or some other kind of quantum dots.

Sequences suitable for making probes/primers for the detection of their corresponding biomarkers include those that are identical or complementary to all or part of genes or SEQ ID NOs: 1, 3, 4, and 6 described herein.

The use of a probe or primer of between 13 and 100 nucleotides, particularly between 17 and 100 nucleotides in length, or in some aspects up to 1-2 kilobases or more in length, allows the formation of a duplex molecule that is both stable and selective. Molecules having complementary sequences over contiguous stretches greater than 20 bases in length may be used to increase stability and/or selectivity of the hybrid molecules obtained. One may design nucleic acid molecules for hybridization having one or more complementary sequences of 20 to 30 nucleotides, or even longer where desired. Such fragments may be readily prepared, for example, by directly synthesizing the fragment by chemical means or by introducing selected sequences into recombinant vectors for recombinant production.

In another embodiment, the probes/primers for a gene are selected from regions which significantly diverge from the sequences of other genes. Such regions can be determined by checking the probe/primer sequences against a human genome sequence database, such as the Entrez database at the NCBI. One algorithm suitable for this purpose is the BLAST algorithm. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold. These initial neighborhood word hits act as seeds for initiating searches to find longer HSPs containing them. The word hits are then extended in both directions along each sequence to increase the cumulative alignment score. Cumulative scores are calculated using, for nucleotide sequences, the parameters M (reward score for a pair of matching residues; always >0) and N (penalty score for mismatching residues; always <0). The BLAST algorithm parameters W, T, and X determine the sensitivity and speed of the alignment. These parameters can be adjusted for different purposes, as appreciated by one of ordinary skill in the art.

In one embodiment, quantitative RT-PCR (such as TaqMan, ABI) is used for detecting and comparing the levels of RNA transcripts in biological samples. Quantitative RT-PCR involves reverse transcription (RT) of RNA to cDNA followed by relative quantitative PCR (RT-PCR).

Yet another method for detecting RNA involves microarray. A microarray consists of an array of thousands of microscopic spots of oligonucleotides containing a specific sequence that are used as probes to hybridize a cDNA or cRNA sample (called target) under high-stringency conditions. Probe-target hybridization can be detected and quantified by fluorescence-based detection of fluorophore-labeled targets to determine relative abundance of nucleic acid sequences in the target. In standard microarrays, the probes are attached to a solid surface by a covalent bond to a chemical matrix. The solid surface can be glass or a silicon chip, or microscopic beads. Microarrays can be used to detect DNA (as in comparative genomic hybridization) or detect RNA (most commonly as cDNA after reverse transcription) that may or may not be translated into proteins.

Other techniques, for example, transcriptome-based techniques may be used in evaluation of RNA, as would be known to those of skill in the art.

In some embodiments, the present disclosure provides a method of overcoming resistance or restoring sensitivity to a therapy targeting the AR signaling axis in a subject suffering from prostate cancer. In some embodiments, the method comprises administering to the subject an effective amount of at least one composition or a pharmaceutical composition effective in restoring expression levels of PRAC1. In some embodiments, the subject has been identified as resistant to a therapy targeting the AR signaling axis by a method comprising: obtaining one or more biological samples from the subject; measuring the expression level of at least one biomarker gene comprising PRAC1 in the one or more biological samples; comparing the expression levels of the at least one biomarker gene comprising PRAC1 in the one or more biological sample obtained from the subject with an expression level of PRAC1 in a reference/control sample. In an embodiment, a lower expression of PRAC1 (PRAC1^{low}) in the one or more biological sample obtained from the subject relative to the reference/control sample identifies the subject as resistant to a therapy targeting the AR signaling axis.

In some embodiments, the at least one composition effective in restoring expression levels of PRAC1 comprises an isolated peptide that binds to the ligand binding domain of an androgen receptor and/or an estrogen receptor. In some embodiments, the isolated peptide comprises an amino acid sequence at least about 90%, 80%, 70%, 60%, 50% identical to the amino acid sequence of wild-type full-length PRAC1 protein. In some embodiments, the isolated peptide comprises the sequence set forth in SEQ ID NO:3. In some embodiment, the isolated peptide comprises an amino acid sequence at least about 90%, 80%, 70%, 60%, 50% identical to the amino acid sequence set forth in SEQ ID NO:3. In some embodiments, the isolated peptide consists of the amino acid sequence set forth in SEQ ID NO: 2. In some embodiments, the isolated peptide comprises 20, 19, 18, 17, 16, 15, 14, 13, 12, 11, 10 amino acid residues of the C-terminus of the wild-type PRAC1 protein. In some embodiments, the isolated peptide comprises the sequence set forth in SEQ ID NO:9. In some embodiments, the isolated peptide consists of the sequence set forth in SEQ ID NO:37. Also disclosed herein are derivatives or pharmaceutically acceptable salts of the isolated peptides disclosed herein.

In some embodiments, the composition is effective in restoring expression levels of PRAC1 to equal to or more than the expression levels of PRAC1 relative to a control/reference sample.

In some embodiments, the peptide/peptidomimetics, derivatives, or portions thereof disclosed herein are effective in suppressing growth of a malignant condition. In some embodiments, the malignant condition is metastatic castration resistant prostate cancer, uterine cancer, ovarian cancer, or breast cancer. In some embodiments, the peptides/peptidomimetics or compositions disclosed herein are administered to a subject in need thereof alone or in combination with at least one other treatment modality or agent. In some embodiments, wherein the condition is prostate cancer the at least one other treatment modality or agent can include therapies that target the androgen receptor (AR) signaling axis including, but not limited to, leuprorelin (leuprolide), goserelin, triptorelin, histrelin, buserelin, and degarelix, flutamide, nilutamide, bicalutamide, enzalutamide, apalutamide, darolutamide, galeterone, abiraterone acetate, seviteronel, and the like. Therapies targeting Poly (ADP-ribose) polymerase (PARP) including but not limited to, olaparib, rucaparib, niraparib, talazoparib, and the like. Therapies directed against cell surface targets including, but not limited to, IMMU-132 (sacituzumab-govitecan), trastuzumab-DXd, a PSMA specific antibody conjugated to monomethyl auristatin E (PSMA-MMAE), Dato-DXd, and the like. Therapies involving radioligands including but not limited to ¹⁷⁷Lu-PSMA-617, radium-223, and the like. Therapies involving chemotherapeutic drugs, including but not limited to docetaxel, cabazitaxel, mitoxantrone, estramustine, carboplatin, and the like. Therapies that involve external beam radiation and/or brachytherapy, and therapies that involve surgery. Therapies that involve immunotherapy, including but not limited to Sipuleucel-T, pembrolizumab, ipilimumab, nivolumab, atezolizumab, durvalumab, avelumab, and the like.

For breast, ovarian, and/or uterine cancers the combined therapies that can include, for example, hormonal therapies, including but not limited to tamoxifen, toremifene, fulvestrant anastrozole, exemestane, letrozole, goserelin, leuprolide, and the like. Therapies involving chemotherapies, including but not limited to, cyclophosphamide, methotrexate, 5-fluorouracil, doxorubicin, paclitaxel, docetaxel, epirubicin, and the like. Targeted therapies, including but not limited to abemaciclib, alpelisib, palbociclib, ribociclib, everolimus, olaparib, rucaparib, talazoparib, lapatinib, neratinib, bevacizumab, and the like. Therapies directed against cell surface antigen, including but not limited to, Trastuzumab, Pertuzumab, Ado-trastuzumab emtansine, trastuzumab-deruxtecan, IMMU-132 (sacituzumab-govitecan), trastuzumab-DXd, Dato-DXd, and the like. Therapies involving external beam radiation, brachytherapy, and/or surgery. Immunotherapies, including but not limited to pembrolizumab, ipilimumab, nivolumab, atezolizumab, durvalumab, avelumab, and the like.

In some embodiments, the at least one other treatment modality or agent comprises an additional therapeutic agent. In some embodiments, the additional therapeutic agent and the isolated peptides/peptidomimetics or compositions as provided herein are formulated into a single therapeutic composition, the isolated peptides/peptidomimetics or compositions and the additional agent are administered simultaneously. In some embodiments, the isolated peptides/peptidomimetics or compositions and the additional agent are separate from each other, e.g., each is formulated into a separate therapeutic composition, and the polypeptides/peptidomimetics or compositions and the additional agent are administered simultaneously, or the polypeptides/peptidomimetics or compositions and the additional agent are administered at different times during a treatment regimen. For example, the polypeptides/peptidomimetics or compositions is administered prior to the administration of the additional agent, the polypeptides/peptidomimetics or compositions is administered subsequent to the administration of the additional agent, or the polypeptides/peptidomimetics or compositions and the additional agent are administered in an alternating fashion. As described herein, the polypeptides/peptidomimetics or compositions and additional agent are administered in single doses or in multiple doses.

The present disclosure also includes a method for producing the PRAC1 peptides/peptidomimetics disclosed herein. The PRAC1 peptides/peptidomimetics disclosed herein can be produced by chemical synthesis or DNA recombinant technology. The principles of chemical synthesis of polypeptides are well known in the art, and general texts in this area can be consulted. An exemplary method of producing the peptide by DNA recombinant technology comprises: step 1 of culturing a host cell harboring the nucleic acid molecule encoding the PRAC1 peptide, a derivative, or a portion thereof or the vector comprising the nucleotide sequence of the PRAC1 peptide, a derivative, or a portion thereof or a cell expressing the PRAC1 peptide, a derivative, or a portion thereof; and/or step 2 of recovering the PRAC1 peptide, a derivative, or a portion thereof from the cultures obtained in step 1. An operation, such as fractionation, chromatography, or purification, known to a person skilled in the art can be applied to step 2. The method can also include further modifying the recovered and/or purified PRAC1 peptide, a derivative, or a portion thereof, to increase stability, enhance cell-penetration (delivery), increase bioactivity, and/or affinity for the target moieties as disclosed herein.

Nucleic acid molecules encoding full-length PRAC1, a portion, or a derivative thereof can be obtained by total synthesis or by modification of nucleic acid encoding full-length PRAC1. Methods for constructing synthetic genes are well known in the art, see Brown et al., Methods in Enzymology, Academic Press, NY Volume 68, pages 109-151. A DNA sequence encoding the peptide of the present disclosure can be designed based on the amino acid sequence, and Model 3400 DNA synthesizer or ABI3900 high throughput nucleic acid synthesizer (Applied Biosystems Inc, 850 Lincoln Center Drive, Foster City CA 94 404). A DNA having the sequence itself can be produced using a normal DNA synthesizer. The nucleic acid molecule encoding the peptides disclosed herein can comprise additional sequences encoding for amino acid motifs that allow for stable expression or accumulation of the peptide in a host cell, enhanced delivery to a cell or a cellular compartment, increase the expression level, and/or facilitate purification after production. Such additional amino acids can be easily cleaved to produce the active/mature PRAC1 peptide upon purification from the host cell.

Also provided are expression vectors comprising the polynucleotides disclosed herein. Various expression vectors that can be used for transformation in prokaryotic cells and eukaryotic cells, general methods in this region can be referred to. (Maniatis et al. (1989) Molecular Cloning; A Laboratory Manual, Cold Springs Harbor Laboratory Press, NY Volumes 1-3 and The Promega Biological Research Products Catalog and The Stratagene Cloning Systems Catalog). In some embodiments, the polynucleotide is operably linked to an expression control sequence. In some embodiments, the expression vector is capable of delivering polynucleotide to a prostate cancer cell. In some embodiments the expression vector is a viral vector. In some embodiments, the viral vector is a lentiviral vector or a retroviral vector.

Host cells can be either eukaryotic cells or prokaryotic cells. Techniques for transforming host cells are well known in the art and can be found in general citations such as Maniatis et al., Prokaryotic host cells generally preferred as these produce more protein at a high rate and are easier to culture. Peptides expressed in bacterial expression systems characteristically aggregate into particles or inclusion bodies that contain high levels of overexpressed protein/peptide. Such typically aggregated proteins are solubilized, denatured, and refolded using techniques well known in the art. See Protein Folding, Kreuger et al. (1990) pages 136-142, edited by Gierasch and King, American Association for Advancement of Science Publication.

Further provided herein is a pharmaceutical composition. In some embodiments, the pharmaceutical composition comprises at least one of the isolated peptides disclosed herein and/or a derivative thereof. In some embodiments, the pharmaceutical composition comprises the polynucleotide encoding the at least one isolated polypeptide disclosed herein. In some embodiment, the pharmaceutical composition comprises the vectors disclosed herein.

In some embodiments, the pharmaceutical composition is effective in restoring PRAC1 expression and/or in treating a steroid hormone driven cancer. In some embodiments, the pharmaceutical composition is effective in suppressing growth of prostate cancer, uterine cancer, ovarian cancer, and or breast cancer. In some embodiments, the pharmaceutical composition is effective in restoring androgen sensitivity in prostate cancer and/or estrogen sensitivity in uterine, ovarian and/or breast cancer. The isolated peptides/peptidomimetics of the present disclosure can be formulated into a pharmaceutical composition/therapeutic agent by combining with a pharmaceutically acceptable carrier, diluent, excipient, or absorption enhancer. Absorption enhancers include, for example, chelating agents (e.g., EDTA, citrate, salicylate), surfactants (e.g., sodium dodecyl sulfate (SDS)), non-surfactants (e.g., unsaturated cyclic urea), and bile acid salts (for example, sodium deoxycholate, sodium taurocholate). Such a pharmaceutical composition/therapeutic agent can be produced by a well-known method in the pharmaceutical field. In an exemplary embodiment, the pharmaceutical compositions/therapeutic agent according to the present disclosure can include an isolated peptide/peptidomimetic of the disclosure, along with a suitable carrier. These pharmaceutical compositions can be included in kits, such as, for example, diagnostic kits.

The therapeutic agents, the isolated peptides/peptidomimetics, or pharmaceutical compositions disclosed herein can be administered to an individual or subject (such as human) via various routes, including, for example, parenteral, intravenous, intraventricular, intra-arterial, intraperitoneal, intrapulmonary, oral, inhalation, intravesicular, intramuscular, intra-tracheal, subcutaneous, intraocular, intrathecal, transmucosal, and transdermal. In some embodiments, sustained continuous release formulation of the therapeutic agent may be used. In some embodiments, the therapeutic agent is administered intravenously. In some embodiments, the therapeutic agent is administered intraportally. In some embodiments, the therapeutic agent is administered intraarterially. In some embodiments, the therapeutic agent is administered intraperitoneally. In some embodiments, the therapeutic agent is administered intrathecally. In some embodiments, therapeutic agent is administered through a ported catheter to spinal fluid. In some embodiments, the therapeutic agent is administered intraventricularly. In some embodiments, the therapeutic agent is administered systemically. In some embodiments, the therapeutic agent is administered by infusion. In some embodiments, the therapeutic agent is administered by infusion through implanted pump. In some embodiments, the therapeutic agent is administered by a ventricular catheter. In some embodiments, the therapeutic agent is administered through a port or portacath. In some embodiments, the port or portacath is inserted into a vein (such as jugular vein, subclavian vein, or superior vena cava).

The therapeutic agents, the isolated peptides/peptidomimetics, or pharmaceutical compositions disclosed herein can be formulated for any of the above modes of administration. In certain embodiments the peptides can be formulated as a controlled release formulation, a time release formulation, an osmotic-controlled release formulation, a microemulsion, a microsphere, a liposome, a nanoparticle, a patch, pump, drug depot, and the like. The therapeutic agents, isolated peptides/peptidomimetics, or pharmaceutical compositions can be formulated with a cationic or non-cationic lipid. In any of the above formulations, the additional active and/or inactive agents can be included, such as, for example, excipients, carrier agents, delivery agents, and the like. These can include, for example, physiologically compatible salts, sugars, bulking agents, surfactants, buffers, and the like.

The present disclosure also provides a novel therapeutic approach for advanced prostate cancer. The present disclosure provides synthetic PRAC1 peptides which are known to bind to androgen receptor (AR) and suppress prostate cancer growth in vitro and in vivo. In addition, the present disclosure demonstrates that the synthetic PRAC1 peptides also bind to and inhibit the interaction of an estrogen receptor with one or more of its co-factors and ligand. This interaction is expected to suppress uterine, ovarian, and/or breast cancer growth in vitro and in vivo.

In some embodiments, provided here is a method of treating a steroid hormone driven cancer in a subject. In some embodiments, the steroid hormone driven cancer is an advanced prostate cancer in a subject. In some embodiments, the steroid hormone driven cancer is uterine, ovarian, and/or breast cancer. In some embodiments, the method comprises: administering to the subject an effective amount of an isolated peptide/peptidomimetic or a pharmaceutical composition disclosed herein. In some embodiments, the isolated peptide/peptidomimetic binds the ligand binding domain of the androgen receptor (AR) and/or estrogen receptor (ER). In some embodiments, the method is effective in suppressing prostate cancer, uterine cancer, ovarian cancer, and/or breast cancer growth. In some embodiments, the advanced prostate cancer is positive for androgen receptor. In some embodiments, the advanced prostate cancer is metastatic castration resistant prostate cancer.

In some embodiments, the method of treating a steroid hormone driven cancer, such as, for example, an advanced prostate cancer, uterine cancer, ovarian cancer, and/or breast cancer comprises administering to the subject a composition effective in restoring expression levels of PRAC1. In some embodiments, the composition effective in restoring PRAC1 expression levels comprises an isolated peptide/peptidomimetic or a pharmaceutical composition of the present disclosure. In some embodiments, the restoring expression levels of PRAC1 comprises an expression level of PRAC1 equal to the control/reference sample or more than relative to the control/reference sample. In some embodiments, the prostate cancer is positive for androgen receptor. In some embodiments, the uterine cancer, ovarian cancer, and/or breast cancer is positive for estrogen receptor. In some embodiments, the prostate cancer, uterine cancer, ovarian cancer, and/or breast cancer is a metastatic cancer. In certain embodiments, the prostate cancer is a castration resistant prostate cancer. In some embodiments, the subject is identified as resistant to a therapy that targets the AR signaling axis. In some embodiments, the subject is identified as resistant to a therapy targeting the AR signaling axis by a method comprising: obtaining one or more biological samples from the subject; measuring the expression level of at least one biomarker gene comprising PRAC1 in the one or more biological samples; comparing the expression levels of the at least one biomarker gene comprising PRAC1 in the one or more biological sample obtained from the subject with an expression level of PRAC1 in a reference/control sample. In some embodiments, a lower expression of PRAC1 (PRAC1^{low}) in the one or more biological sample obtained from the subject relative to the reference/control sample identifies the subject as resistant to a therapy targeting the AR signaling axis.

The following examples are included to demonstrate preferred embodiments of the disclosure. It should be appreciated by those of skill in the art that the techniques disclosed in the examples which follow represent techniques discovered by the inventor to function well in the practice of the disclosure, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the disclosure.

### Example 1

### PRAC1 is a Biomarker of Response to Hormonal Therapies in Prostate Cancer

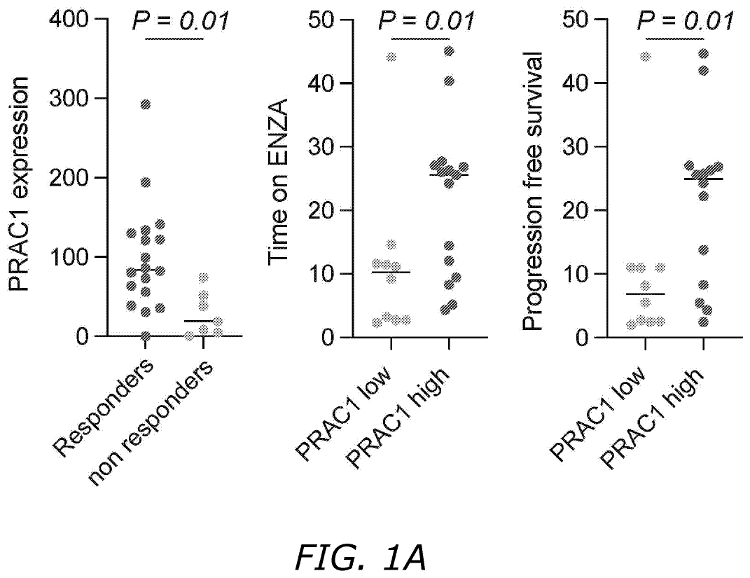

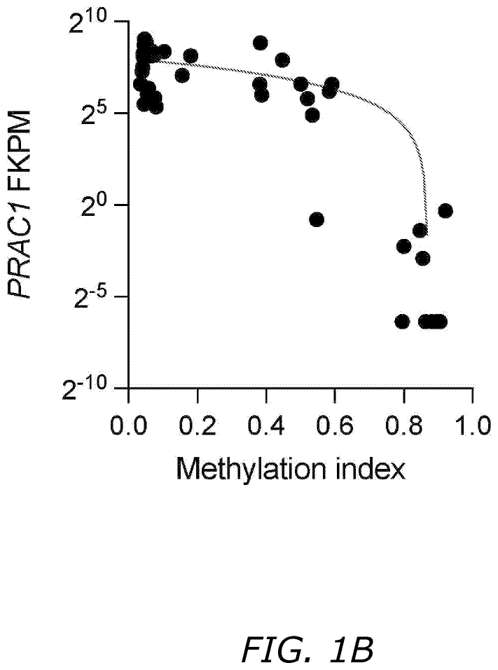

Given the foregoing observation that PRAC1 expression is associated with response to hormonal therapies, the inventors assessed publicly available RNA-sequencing data to determine if PRAC1 expression levels in metastatic prostate cancer are different between patients who respond and those who do not respond to androgen receptor signaling inhibitor (ARSI) enzalutamide therapy. Publicly available RNA-sequence data (Alumkal et al. Proc. Nat'l. Acad. Sci. USA 117(22)12315-12323, 2020) were analyzed using established data analysis pipelines (Patel et al., J. Pathol. 262(1):105-120, 2024; doi: 10.1002/path.6216. Epub. 2023 Oct. 18; and Sayar et al., JCI Insight 8(7):e162907, 2023), and PRAC1 expression was determined. Tumors were stratified into high and low PRAC1 expressors, and expression data were correlated with available clinical response data. (FIG. 1A).

PRAC1 expression was determined by RNA-seq in 44 prostate cancer patient-derived xenograft models (Laberecque et al., J. Clin. Invest. 129(10):4492-4505, 2019). PRAC1 gene methylation (hg19 CHr.17:46797568-46799745) was determined using Infinium EPIC assays (Patel et al., J. Pathol. 262(1):105-120, 2024; doi: 10.1002/path.6216. Epub. 2023 Oct. 18). Methylation beta-values were correlated with PRAC1 expression (FPKM; Fragments per Kilobase per Million mapped fragments), demonstrating a tight association between PRAC1 expression and methylation (R-squared 0.38; P<0.0001). (FIG. 1B).

The results of these studies show that PRAC1 expression is higher in responders compared to non-responders and patients with higher PRAC1 expression show longer progression free survival. Furthermore, patients with low PRAC1 expression show earlier disease progression. Methylation and mRNA expression analyses can be used to stratify patients based on PRAC1 status.

### Example 2

### The First Open Reading Frame of PRAC1 in Involved in Regulating Prostate Cancer Cell Growth

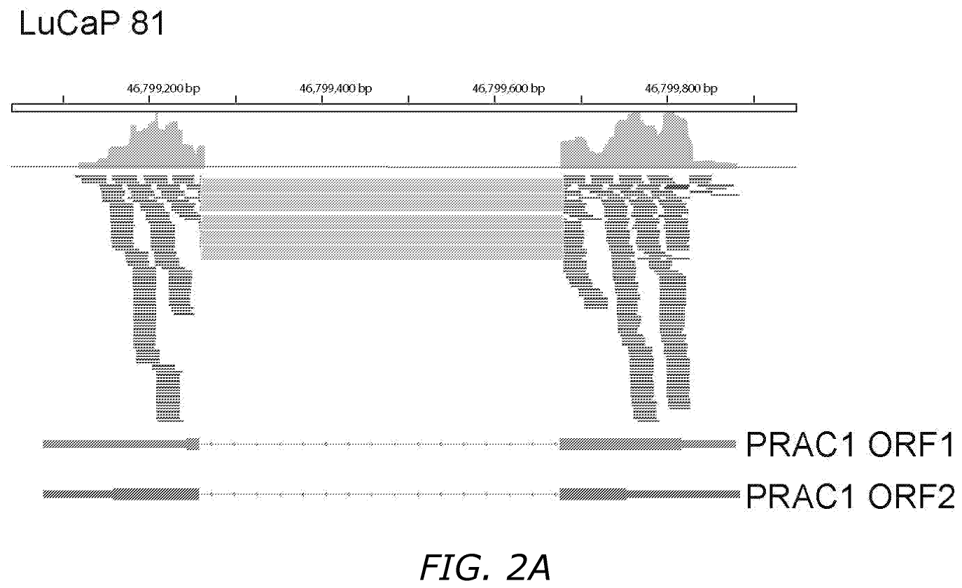

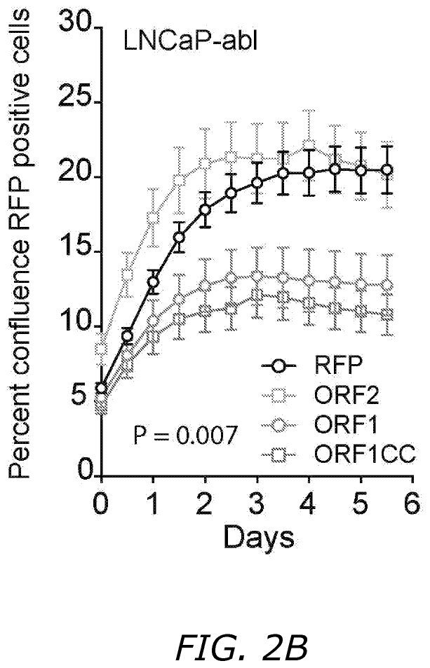

The nature of the biologically active intermediate of the PRAC1 gene is unknown. To determine the operative ORF encoding for PRAC1 protein, reads from polysome-enriched RNA analysis (Schuster et al., Cell Rep. 42(8):112840. doi: 10.1016/j.celrep.2023.112840. Epub 2023 Jul. 28) were analyzed. While all current gene annotation databases list the second ORF (ORF2) of PRAC1 as protein coding, the present ribosomal profiling analysis revealed ribosome entry at the first ORF (ORF1). This suggested that PRAC1 is translated from a previously unannotated translational start site in prostate cancer cells. Notably, ORF1 and ORF2 peptides have different amino acid sequences. (FIG. 2A).

ORF1 full length
SEQ ID NO: 3
MLACKVRQAGFWSPPLQREQRCCAPISQIKDRPILLPPRVLFSLIRKHLL
ORF2 full length
SEQ ID NO: 5
MLCAHFSDQGPAHLTTSKSAFLSNKKTSTLKHLLGETRSDGSACNSGISG
GRGRKIP

To functionally corroborate the effect of different PRAC1 ORFs on prostate cancer growth, expression constructs encoding ORF1, ORF2, and a fully codon-changed ORF1 (ORF1cc) were lentivirally delivered into PRAC1-negative LNCaP-abl prostate cancer cells and were grown in the absence of charcoal stripped medium. Only the expression of ORF1 and ORF1cc showed growth suppression, indicating that ORF1 is functionally active. (FIG. 2B), i.e., expression of the first, but not the second open reading frame results in growth suppression of castration resistant prostate cancer cells

ORF1
(SEQ ID NO: 2)
ATGCTTGCTTGCAAGGTCAGGCAAGCTGGATTCTGGTCCCCACCTTTGCA
GAGAGAACAGCGATGTTGTGCGCCCATTTCTCAGATCAAGGACCGGCCCA
TCTTACTACCTCCAAGAGTGCTTTTCTCTCTAATAAGAAAACATCTACTT
TGA
ORF2
(SEQ ID NO: 4)
ATGTTGTGCGCCCATTTCTCAGATCAAGGACCGGCCCATCTTACTACCTC
CAAGAGTGCTTTTCTCTCTAATAAGAAAACATCTACTTTGAAACATCTAC
TGGGCGAGACCAGGAGTGATGGCTCAGCCTGTAATTCTGGAATTTCGGGA
GGCCGAGGCAGGAAGATTCCTTGA
ORF1cc
(SEQ ID NO: 6)
ATGTTGGCGTGTAAAGTGCGCCAGGCGGGCTTTTGGAGTCCCCCGCTCCA
ACGCGAGCAAAGGTGCTGCGCCCCGATAAGCCAAATAAAAGATAGACCGA
TACTCTTGCCGCCCCGCGTCTTGTTTAGCTTGATCCGCAAGCACTTGTTG
TGA

Defining the operative ORF and identifying the crucial biologically active peptide encoded by PRAC1 provides crucial information for the development of PRAC1 as a therapeutic target and biomarker.

### Example 3

### PRAC1 Binds to the Androgen Receptor and Regulates Transcriptional Outputs

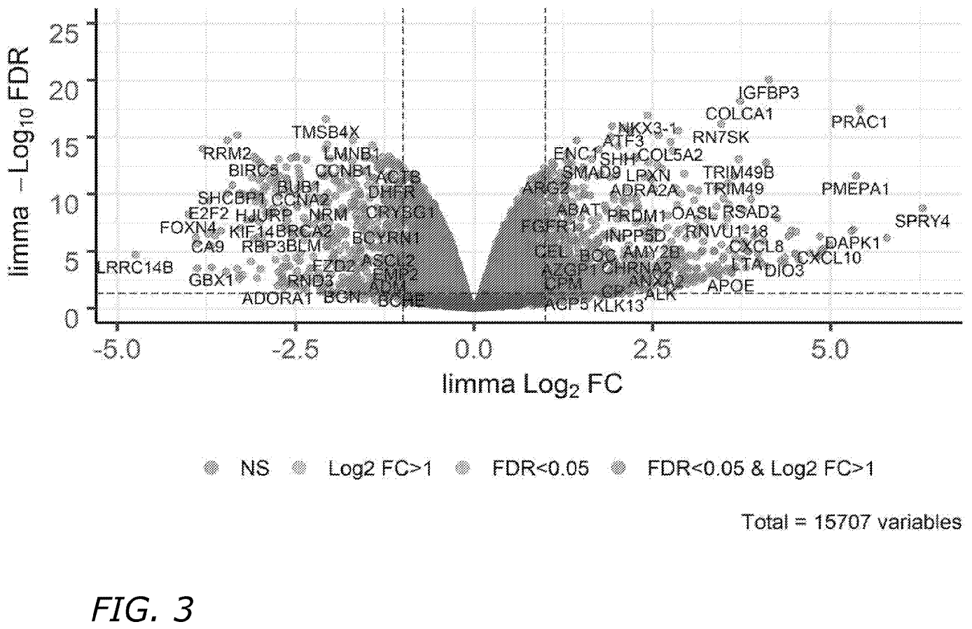

PRAC1 was expressed in PRAC1-negative LNCaP-abl prostate cancer cells using lentiviral delivery. RNA-sequencing was performed on the expressed RNA as previously described (Laberecque et al., J. Clin. Invest. 129(10):4492-4505, 2019). PRAC1 expression resulted in profound transcriptional changes with increased expression in pro-differentiation and luminal prostate epithelial gene sets, and repression of cell proliferation-associated gene sets. (FIG. 3). This establishes that PRAC1 is regulating prostate cancer cell differentiation and proliferation at the transcriptional level.

### Example 4

### PRAC1 Regulates the Sensitivity to Hormonal-Directed Therapies

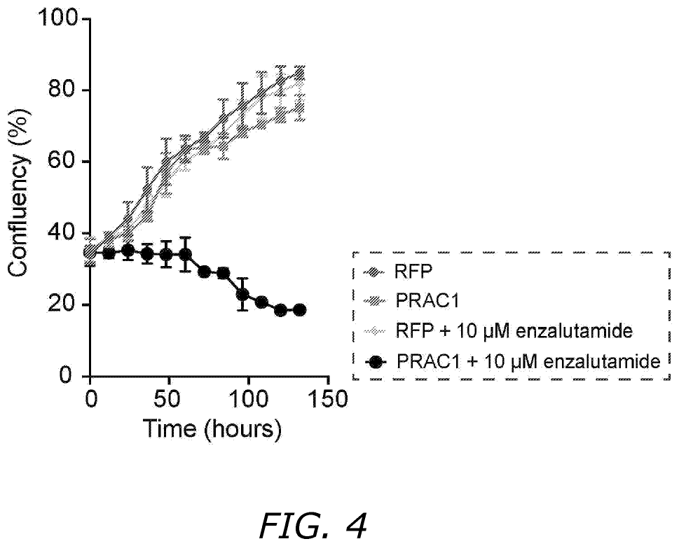

Resistance to ARSI therapy is a major driver of PC related mortality. In this example, PRAC1-negative LNCaP-abl cells were grown in fully supplemented growth media (RPMI plus 10% FBS) in the absence or presence of 10 μM enzalutamide. Cell proliferation was determined longitudinally by live cell imaging using a Cytation™ 5 imaging instrument. While LNCaP-abl cells transduced with a control vector (RFP) are resistant to enzalutamide, re-expression of PRAC1 in LNCaP-abl cells results in sensitization to enzalutamide and significant growth suppression. These results demonstrate that PRAC1 loss is a key mediator of androgen receptor signaling inhibitor (ARSI) resistance in CRPC. Restoring PRAC1 function can result in re-sensitization of ARSI therapy (FIG. 4).

### Example 5

### Synthetic Cell Penetrable PRAC1 Peptide Mimetics can Suppress Prostate Cancer Growth in Vitro and In Vivo

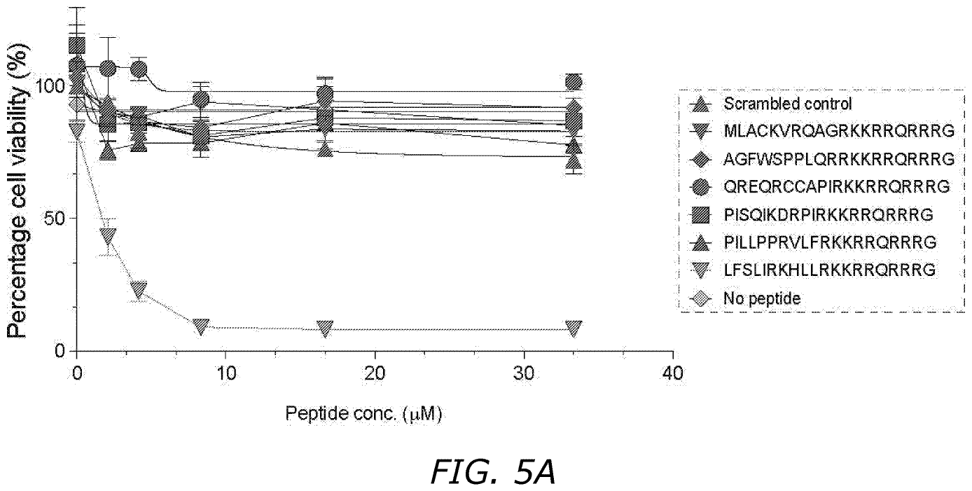

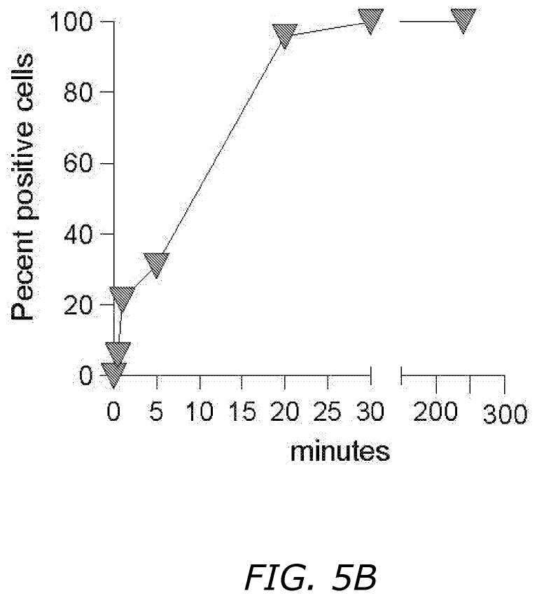

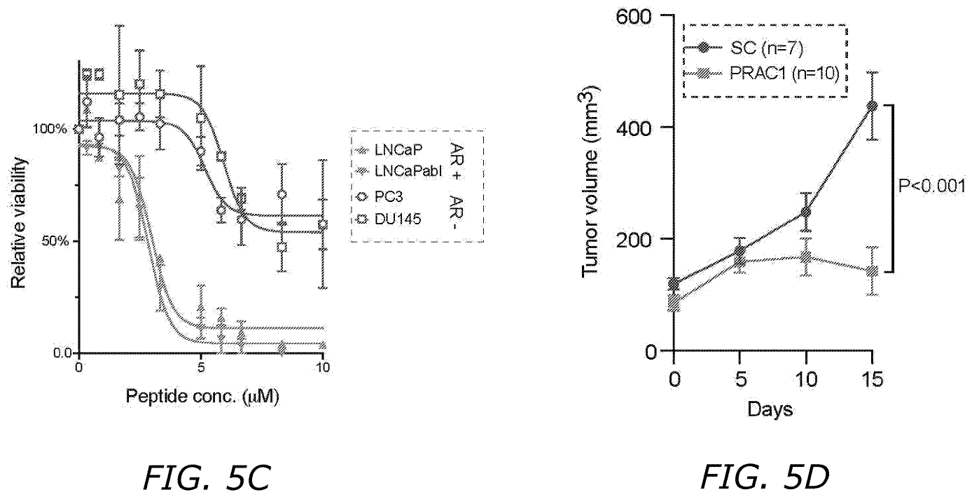

To determine if exogenously delivered PRAC1 peptides affect prostate cancer growth, cell-permeable peptides were generated by synthesizing overlapping 10-mer PRAC1 peptide fragments conjugated to the arginine-rich region of TAT (RKKRRQRRRG; SEQ ID NO:7), a peptide sequence known to result in cellular uptake. PRAC1-negative LNCaP-abl cells were grown and exposed to increasing concentrations of the cell-permeable peptides, and cell viability was measured after 72 hours using CellTiter-Blue® (Patel et al., Cancer Res. Commun. 2(5):277-285 2022. doi: 10.1158/2767-9764.crc-21-0156. Epub 2022 May 2). In vitro assays showed that only the C-terminus of PRAC1 (PRAC1-F6; LFSLIRKHLLRKKRRQRRRG; SEQ ID NO:17) linked to the cell permeable TAT peptide is biologically active in reducing cell viability (FIG. 5A). The PRAC-F6 peptide was further tested for cell permeability. The PRAC1 C-terminal peptide (PRAC-F6 was N-terminally labeled with fluorescein isothiocyanate (FITC) and incubated for the indicated times. Peptide uptake was measured by flow cytometry and showed rapid cellular internalization. (FIG. 5B)

Cell viability of androgen receptor negative and androgen receptor positive cells was tested in vitro. The androgen positive cell lines LNCaP and LNCaP-abl and the androgen receptor negative cell lines PC3 and DU145 were incubated with the PRAC-F6, carboxy terminal peptide including the TAT cell penetration peptide. Cell viability was measured after 72 hours using CellTiter-Blue®. It should be noted that only AR-expressing cell lines show a profound loss of viability (FIG. 5C).

The peptide was also tested in an in vivo in prostate cancer xenograft model. Male NOD SCID gamma (NSG) mice were chemically castrated by subcutaneous injection of degarelix. Mice were then engrafted with PRAC1-negative human prostate cancer LAPC4-CR cells. When tumors reached palpable size, PRAC1 peptide PRAC-F6 or a scrambled control peptide (sc) were delivered intraperitoneally daily. Tumor size was measured by caliper. The tumor growth difference between the PRAC1 peptide treated mice versus the sc peptide treated mice is notable and showed a suppression of tumor growth (FIG. 5D).

These data demonstrate and provide a novel therapeutic approach for advanced prostate cancer. These data show that synthetic peptides which are known to bind to AR suppress prostate cancer growth in vitro and in vivo.

### Example 6

### Structural Requirements for AR-PRAC1 Interaction

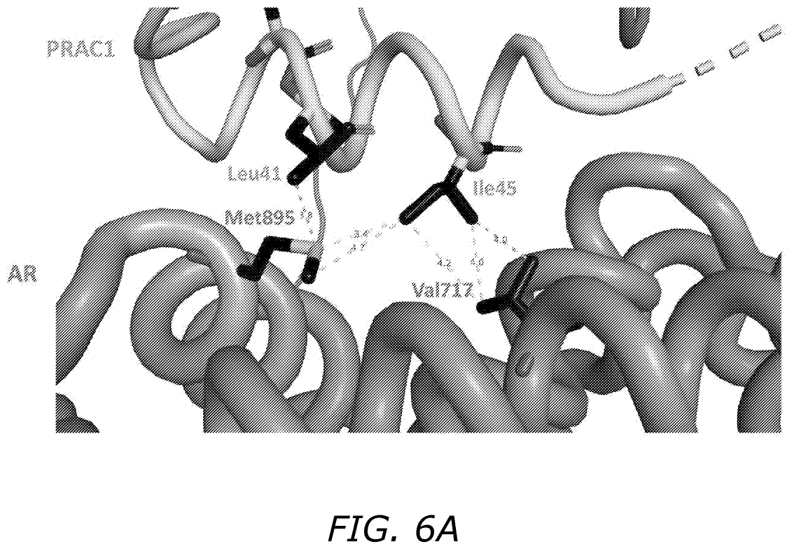

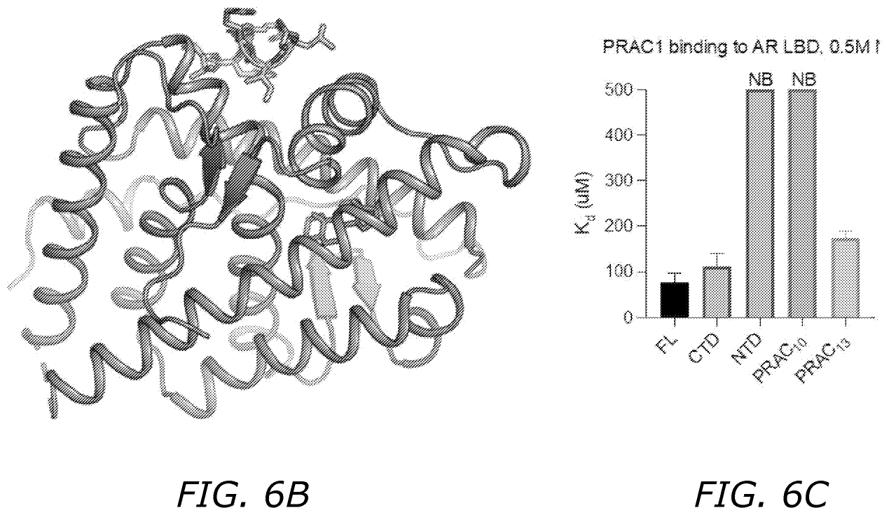

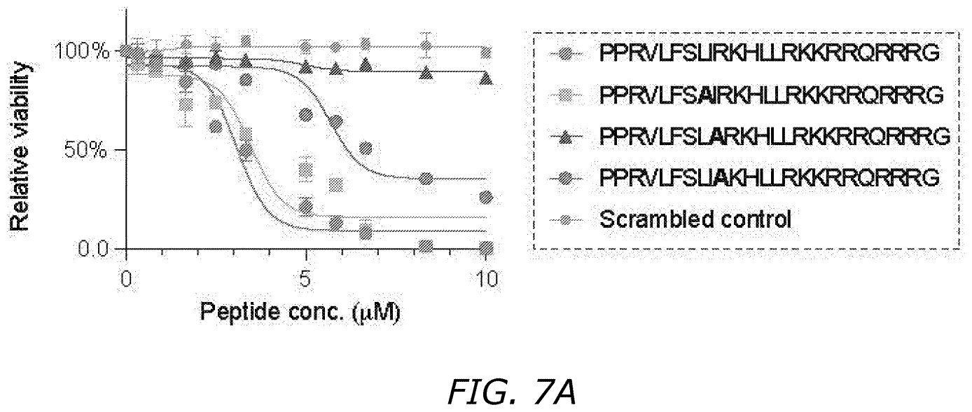

An AlphaFold model showing the interaction of PRAC1 to the androgen receptor (AR) was created using the methods of Jumper et al. (Nature 596(7873):583-589, 2021. doi: 10.1038/s41586-0319-2. Epub 2021 Jul. 15) FIG. 6A. FIG. 6B is the crystal structure analyses (similar to Wasmuth et al., Nature 511(7510):435-439, 2014. doi: 10.1038/nature 13406. Epub 2014 Jul. 6) of the AR ligand binding domain bound to PRAC1 peptide LFSLIRKHLL (SEQ ID NO:37). These analyses reveal that the AR ligand binding domain binds to PRAC1 revealing the atomic contacts between the PRAC1 C-terminus with the AR. Efforts to develop more potent AR inhibitors have been hampered by challenges isolating highly pure, specific protein preparations of full-length AR. The inventors have isolated the means to do so (Wasmuth et al, Proc. Nat'l. Acad. Sci. USA 117(15):8584-8592, 2020. doi: 10.1073/pnas.1922159117. Epub 2020 Mar. 27; Wasmuth et al, Mol. Cell. 82(1):2021-2031.e5, 2022. doi: 10.1016/j.molcel.2022.03.035. Epub 2022 Apr. 20) and have demonstrated that PRAC1 specifically binds and alters full-length AR's DNA binding activity.

Recombinant protein-based assays demonstrate that PRAC1 specifically binds the AR LBD even under highly stringent conditions (0.5M NaCl) by surface plasmon resonance (FIG. 6C).

### Example 7

### Systematic Analysis of Crucial Amino Acids Required for PRAC1 Function

LNCaP-abl cells were incubated with increasing concentrations of PRAC1 peptides, and cell viability was determined after 72 hours using CellTiter-Blue®. Notably, PRAC1 peptide alanine substitutions using a systematic alanine scan showed differential activity on cell viability. The peptides tested included:

(SEQ ID NO: 19)
APRVLFSLIRKHLLRKKRRQRRRG,
(SEQ ID NO: 20)
PARVLFSLIRKHLLRKKRRQRRRG,
(SEQ ID NO: 21)
PPAVLFSLIRKHLLRKKRRQRRRG,
(SEQ ID NO: 22)
PPRALFSLIRKHLLRKKRRQRRRG,
(SEQ ID NO: 23)
PPRVAFSLIRKHLLRKKRRQRRRG,
(SEQ ID NO: 24)
PPRVLASLIRKHLLRKKRRQRRRG,
(SEQ ID NO: 25)
PPRVLFALIRKHLLRKKRRQRRRG,
(SEQ ID NO: 26)
PPRVLFSAIRKHLLRKKRRQRRRG,
(SEQ ID NO: 27)
PPRVLFSLARKHLLRKKRRQRRRG,
(SEQ ID NO: 28)
PPRVLFSLIAKHLLRKKRRQRRRG,
(SEQ ID NO: 29)
PPRVLFSLIRAHLLRKKRRQRRRG,
(SEQ ID NO: 30)
PPRVLFSLIRKALLRKKRRQRRRG,
(SEQ ID NO: 31)
PPRVLFSLIRKHALRKKRRQRRRG,
and
(SEQ ID NO: 32)
PPRVLFSLIRKHLARKKRRQRRRG.

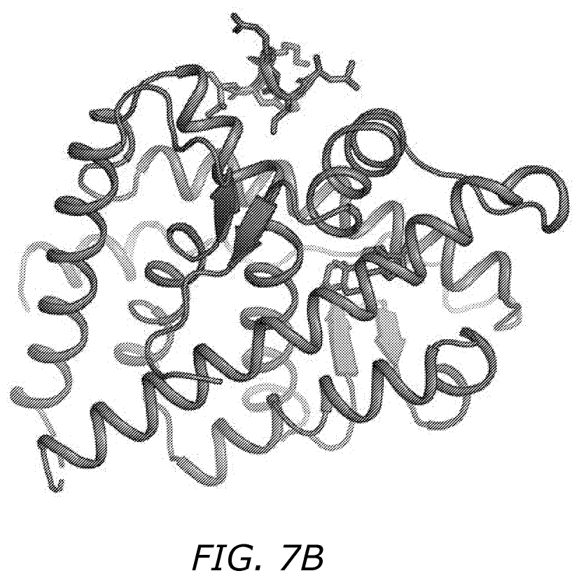

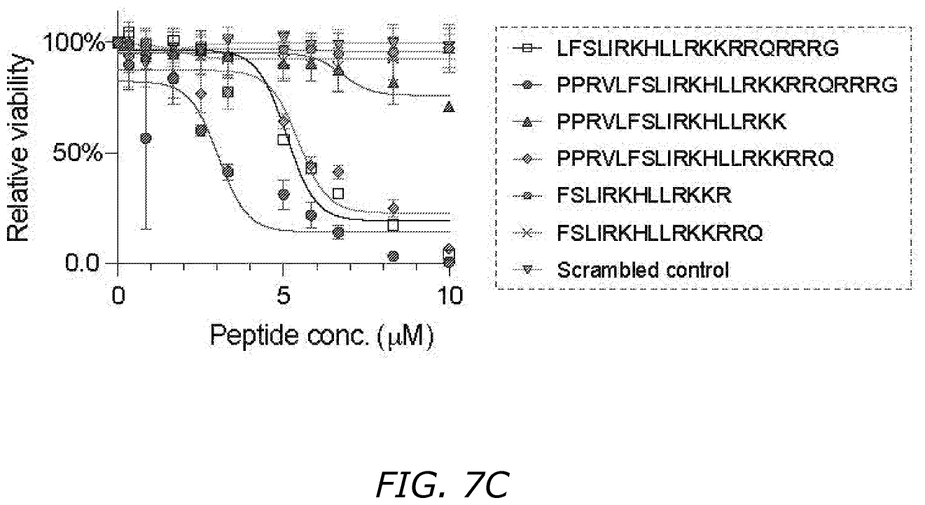

While the active peptide (PPRVLFSLIRKHLLRKKRRQRRRG; SEQ ID NO: 18) exhibited profound anti-tumor activity, as isoleucine-to-alanine substitution mutant (19A; PPRVLFSLARKHLLRKKRRQRRRG; SEQ ID NO:27) showed no effect on cell viability. FIG. 7A. Furthermore, an arginine-to-alanine mutant (R10A; PPRVLFSLIAKHLLRKKRRQRRRG; SEQ ID NO:28) showed attenuated activity on cell viability.

FIG. 7B shows the crystal structure of the AR LBD bound to PRAC1 was also resolved for the arginine rich region containing PRAC1 peptide (PRVLFSLIRKHLLRKKRRQ; amino acid residues 2 to 20 of SEQ ID NO:34) and the network of interactions between the two proteins is identical to those observed in the crystal structure of the AR LBD bound to PRAC1 peptide LFSLIRKHLL (SEQ ID NO:37). The peptide PPRVLFSLIRKHLL (SEQ ID NO:9) provides the C-terminal PRAC1 amino acid sequence with highlighted key amino acid residues and minimal peptide sequence required for PRAC1 activity in AR positive cell lines.

FIG. 7C shows the cell viability of LNCaP-abl cells exposed to different PRAC1 peptides. LFSKIRKHLLRKKRRQRRRG (SEQ ID NO:17); PPRVLFSLIRKHLLRKKRRQRRRG (SEQ ID NO:20); PPRVLFSLIRKHLLRKK (SEQ ID NO: 33; PPRVRVLESLIRKHLLRKKRRQ (SEQ ID NO:34); FSLIRKHLLRKKR (SEQ ID NO:35); FSLIRKHLLRKKRRQ (SEQ ID NO:36).

### Example 8

### PRAC1 Peptides Show Activity in Estrogen Receptor Positive Breast Cancer

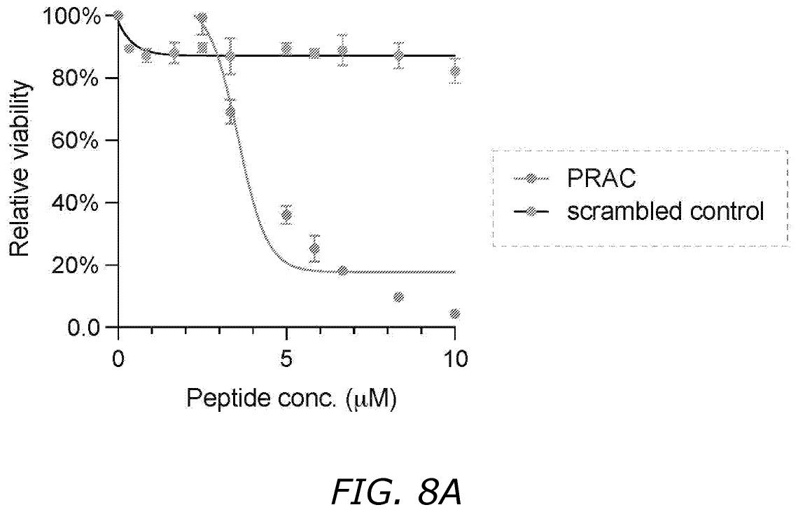

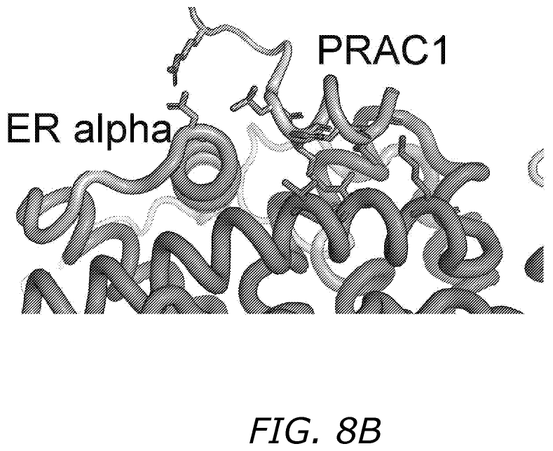

Estrogen and progesterone receptor positive breast cancer cell line MCF7 with PRAC1 peptide PPRVLFSLIRKHLLRKKRRQRRRG (SEQ ID NO:18) results in a dose-dependent decrease in cell viability as determined by CellTiter-Blue® relative to scrambled control (sc) peptide. FIG. 8A. An AlphaFold model as described above as assembled shows the interaction of PRAC1 (SEQ ID NO:3) to the estrogen receptor alpha.

### Example 9

### A Scalable High Throughput Assay to Study AR-PRAC1 Interactions

To identify small molecules that bind to the AR/PRAC1 interface, a scalable TR-FRET assay has been developed that allows the performance of the screens in a 384 well format. The assay combines the time-resolved (TR) measurement of fluorescence with fluorescence resonance energy transfer (FRET). The ligand binding domain (LBD) of the androgen receptor containing a His-tag is expressed in bacteria. Recombinant His-tagged AR is further labeled with Terbium-labeled anti-His-tag antibodies and incubated with FITC-labeled PRACA1 peptides. The binding (i.e., proximity of AR and PRAC1) generates the FRET signal in this assay.

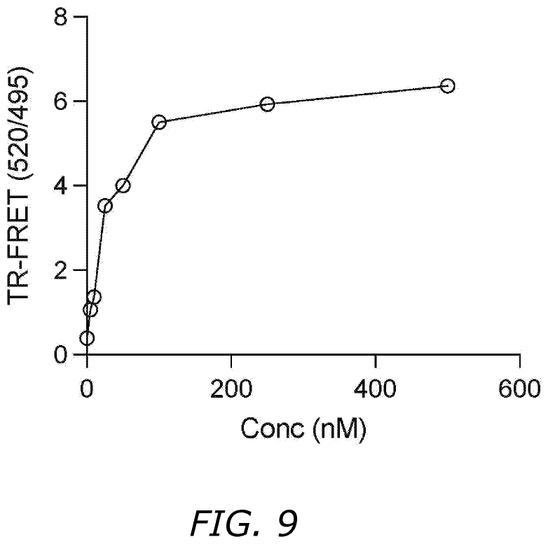

This is the first saleable in vitro assay to determine the interaction of AR and PRAC1. It can be used to test the affinity of modified PRAC1 mimetic peptides and represents a platform technology for screening small molecules that interfere with the AR-PRAC1 interaction. Shown in FIG. 9 is the dose dependent increase in FRET signal with PRAC1 peptide PPRVLFSLIRKHLL (SEQ ID NO:9).

### Example 10

### Sequence and Predicted Structure of Modified PRAC1 Peptides

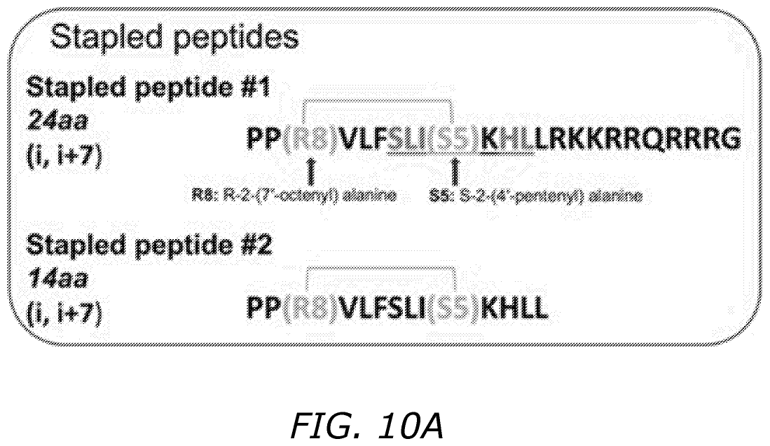

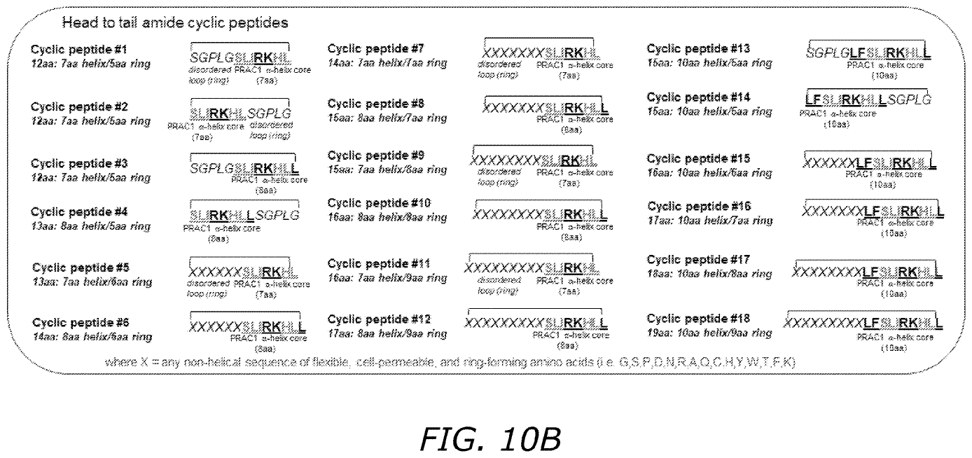

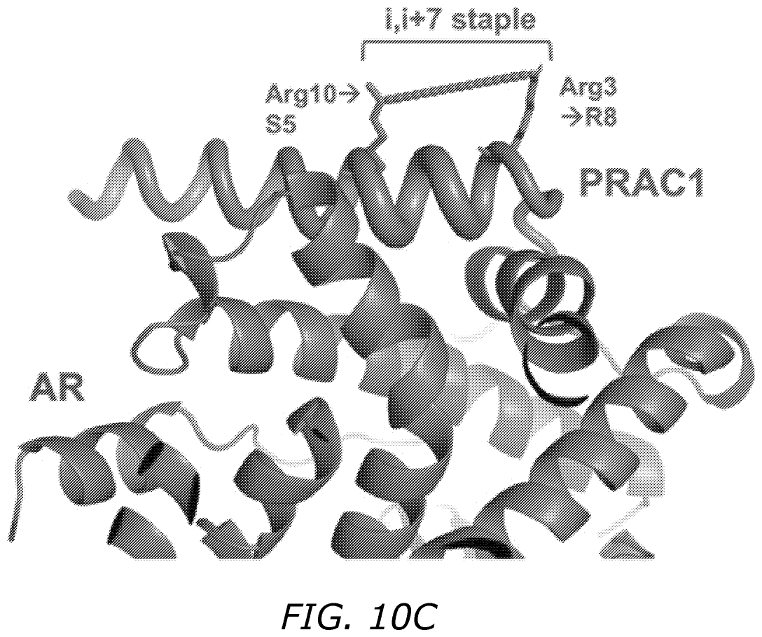

Two structure-guided approaches were taken to further increase PRAC1 peptide stability, affinity for the AR, and bioactivity: introduction of a peptide staple (FIG. 10A) and cyclization of the minimal bioactive sequence identified by the crystal structure (FIG. 10B), in vivo alanine scan, and structural modeling using AlphaFold (FIG. 10C).

These rational designs leverage residues for stapling or cyclization that are outside of the PRAC1 bioactive core and that do not make intra- or intermolecular interactions with AR based on experimental data (crystal structures). The introduction of the i,i+7 staple at Arg3 (R8: R-2-(7′-octenyl) alanine) and Arg10 (S5: S-2-(4′-pentenyl) alanine) stabilize the PRAC1 helix based on a combination of AlphaFold modeling and crystal structures of the AR LBD bound to PRAC1 (LFSLIRKHLL; SEQ ID NO:37 and PRVLFSLIRKHLLRKKRRQRRRG, SEQ ID NO:18) where Arg3 is visible but Arg10 is disordered due to flexibility. The stapled peptides include stapled peptide #1 is PPX³VLFSLIX¹⁰KHLLRKKRRQRRRG, wherein X³ is R8: R-2-(7′-octenyl) alanine and X¹⁰ is S5: S-2-(4′pentenyl) alanine; SEQ ID NO:38) and stapled peptide #2 is PPX³VLFSLIX¹⁰KHLL, wherein X³ is R8::R-2-(7′-octenyl) alanine and X¹⁰ is S5: S-2-(4′pentenyl) alanine; SEQ ID NO:39. Head to tail amide cyclization minimizes peptide size, bypassing the need for the additional solubility motif RKRRQRRRG (SEQ ID NO:7). The various head to tail peptides are depicted in FIG. 10B and include cyclic peptide #1 (S*GPLGSLIRKHL*, wherein * indicates the head to tail amide bond, SEQ ID NO:60), cyclic peptide #2 (S*LIRKHLSGPLG*, wherein * indicates the head to tail amide bond, SEQ ID NO: 61), cyclic peptide #3 (S*GPLGSILRKHLL*, wherein * indicates the head to tail amide bond, SEQ ID NO:62), cyclic peptide #4 (S*LIRKHLLSGPLG*, wherein * indicates the head to tail amide bond, SEQ ID NO:63), cyclic peptide #5 (X*XXXXXSLIRKHL*, wherein * indicates the head to tail amide bond, SEQ ID NO: 44), cyclic peptide #6 (X*XXXXXSLIRKHLL*, wherein * indicates the head to tail amide bond, SEQ ID NO:45), cyclic peptide #7 (X*XXXXXXSLIRKHL*, wherein * indicates the head to tail amide bond, SEQ ID NO:48), cyclic peptide #8 (X*XXXXXXSLIRKHLL*, wherein * indicates the head to tail amide bond, SEQ ID NO: 49), cyclic peptide #9 (X*XXXXXXXSLIRKHL*, wherein * indicates the head to tail amide bond, SEQ ID NO:52), cyclic peptide #10 (X*XXXXXXXSLIRKHLL*, wherein * indicates the head to tail amide bond, SEQ ID NO:53), cyclic peptide #11 (X*XXXXXXXXSLIRKHL*, wherein * indicates the head to tail amide bond, SEQ ID NO: 56), cyclic peptide #12 (X*XXXXXXXXSLIRKHLL*, wherein * indicates the head to tail amide bond, SEQ ID NO:57). Wherein the series of X amino acid residues in each peptide is any non-helical sequence of flexible, cell-permeable, and ring-forming amino acids (i.e., G, S, P, D, N, R, A, Q, C, H, Y, W, T, F, and K).

While illustrative embodiments have been illustrated and described, it will be appreciated that various changes can be made therein without departing from the spirit and scope of the invention.

TABLE 1
DNA and amino acid sequences disclosed herein

PRAC1 sequences
Gene sequence: SEQ ID NO: 1
GTCCTTCCTCTCCTAGCCTAAGGCGTGCAAACAGAGCGCCACTGGGAGGC
TGAAACCTTTAGGCCGATGCTTGCTTGCAAGGTCAGGCAAGCTGGATTCT
GGTCCCCACCTTTGCAGAGAGAACAGCGATGTTGTGCGCCCATTTCTCAG
ATCAAGGACCGGCCCATCTTACTACCTCCAAGAGTGCTTTTCTCTCTAATA
AGAAAACATCTACTTTGAAACATCTACTGGGCGAGACCAGGAGTGATGGC
TCAGCCTGTAATTCTGGAATTTCGGGAGGCCGAGGCAGGAAGATTCCTTG
AGCACAGGAGTTCCAGACCAGCCTGGGCAATGTAGCAAGACGCTGTCTCT
ATTTATACAATAAAATTTTTTTAAAAAAGGAAAAAAAAAAAAAAAAAAA
AAA
ORF1 sequence: SEQ ID NO: 2
ATGCTTGCTTGCAAGGTCAGGCAAGCTGGATTCTGGTCCCCACCTTTGCAG
AGAGAACAGCGATGTTGTGCGCCCATTTCTCAGATCAAGGACCGGCCCAT
CTTACTACCTCCAAGAGTGCTTTTCTCTCTAATAAGAAAACATCTACTTTG
A
ORF1 full length SEQ ID NO: 3
MLACKVRQAGFWSPPLQREQRCCAPISQIKDRPILLPPRVLFSLIRKHLL
ORF2 sequence: SEQ ID NO: 4
ATGTTGTGCGCCCATTTCTCAGATCAAGGACCGGCCCATCTTACTACCTCC
AAGAGTGCTTTTCTCTCTAATAAGAAAACATCTACTTTGAAACATCTACTG
GGCGAGACCAGGAGTGATGGCTCAGCCTGTAATTCTGGAATTTCGGGAGG
CCGAGGCAGGAAGATTCCTTGA
ORF2 full length SEQ ID NO: 5
MLCAHFSDQGPAHLTTSKSAFLSNKKTSTLKHLLGETRSDGSACNSGISGGRG
RKIP
ORFIcc SEQ ID NO: 6
ATGTTGGCGTGTAAAGTGCGCCAGGCGGGCTTTTGGAGTCCCCCGCTCCA
ACGCGAGCAAAGGTGCTGCGCCCCGATAAGCCAAATAAAAGATAGACCG
ATACTCTTGCCGCCCCGCGTCTTGTTTAGCTTGATCCGCAAGCACTTGTTG
TGA
TAT peptide cell penetration peptide
RKKRRQRRG SEQ ID NO: 7 synthetic construct, amino acid
PRAC F6 active peptide SEQ ID NO: 8 (cell penetration
peptide sequence is underlined)
PPRVLFSLIRKHLLRKKRRQRRRG
Key amino acids mediating the interaction between
AR and PRAC1 (underlined and bolded)
PPRVLFSLIRKHLL SEQ ID NO: 9
Minimally active peptide with cell penetration peptide:
SEQ ID NO: 10
LFSLIRKHLLRKKRRQ
Peptides tested:
MLACKVRQAGFWSPPLQREQRCCAPISQIKDRPILLPPRVLFSLIRKHLLRKKR
RQRRRG SEQ ID NO: 11
MLACKVRQAGRKKRRQRRRG SEQ ID NO: 12
AGFWSPPLQRRKKRRQRRRG SEQ ID NO: 13
QREQRCCAPIRKKRRQRRRG SEQ ID NO: 14
PISQIKDRPIRKKRRQRRRG SEQ ID NO: 15
PILLPPRVLFRKKRRQRRRG SEQ ID NO: 16
LFSLIRKHLLRKKRRQRRRG SEQ ID NO: 17
PPRVLFSLIRKHLLRKKRRQRRRG SEQ ID NO: 18
APRVLFSLIRKHLLRKKRRQRRRG SEQ ID NO: 19
PARVLFSLIRKHLLRKKRRQRRRG SEQ ID NO: 20
PPAVLFSLIRKHLLRKKRRQRRRG SEQ ID NO: 21
PPRALFSLIRKHLLRKKRRQRRRG SEQ ID NO: 22
PPRVAFSLIRKHLLRKKRRQRRRG SEQ ID NO: 23
PPRVLASLIRKHLLRKKRRQRRRG SEQ ID NO: 24
PPRVLFALIRKHLLRKKRRQRRRG SEQ ID NO: 25
PPRVLFSAIRKHLLRKKRRQRRRG SEQ ID NO: 26
PPRVLFSLARKHLLRKKRRQRRRG SEQ ID NO: 27
PPRVLFSLIAKHLLRKKRRQRRRG SEQ ID NO: 28
PPRVLFSLIRAHLLRKKRRQRRRG SEQ ID NO: 29
PPRVLFSLIRKALLRKKRRQRRRG SEQ ID NO: 30
PPRVLFSLIRKHALRKKRRQRRRG SEQ ID NO: 31
PPRVLFSLIRKHLARKKRRQRRRG SEQ ID NO: 32
PPRVLFSLIRKHLLRKK SEQ ID NO: 33
PPRVLFSLIRKHLLRKKRRQ SEQ ID NO: 34
FSLIRKHLLRKKR SEQ ID NO: 35
FSLIRKHLLRKKRRQ SEQ ID NO: 36
LFSLIRKHLL SEQ ID NO: 37
PPXVLFSLIXKHLLRKKRRQRRRG, wherein X³ is R-2-(7′-octanyl)
alanine, and X¹⁰ is S-2-(4′-pentenyl) alanine SEQ ID NO: 38
PPXVLFSLIXKHLL, wherein X3 is R-2-(7′-octanyl) alanine,
and X10 is S-2-(4′-pentenyl) alanine SEQ ID NO: 39
Cyclical peptides with head-to-tail amide bond
XXXXXSLIRKHL SEQ ID NO: 40
XXXXXSLIRKHLL SEQ ID NO: 41
SLIRKHLXXXXX SEQ ID NO: 42
SLIRKHLLXXXXX SEQ ID NO: 43
XXXXXXSLIRKHL SEQ ID NO: 44
XXXXXXSLIRKHLL SEQ ID NO: 45
SLIRKHLXXXXXX SEQ ID NO: 46
SLIRKHLLXXXXXX SEQ ID NO: 47
XXXXXXXSLIRKHL SEQ ID NO: 48
XXXXXXXSLIRKHLL SEQ ID NO: 49
SLIRKHLXXXXXXX SEQ ID NO: 50
SLIRKHLLXXXXXXX SEQ ID NO: 51
XXXXXXXXSLIRKHL SEQ ID NO: 52
XXXXXXXXSLIRKHLL SEQ ID NO: 53
SLIRKHLXXXXXXXX SEQ ID NO: 54
SLIRKHLLXXXXXXXX SEQ ID NO: 55
XXXXXXXXXSLIRKHL SEQ ID NO: 56
XXXXXXXXXSLIRKHLL SEQ ID NO: 57
SLIRKHLXXXXXXXXX SEQ ID NO: 58
SLIRKHLLXXXXXXXXX SEQ ID NO: 59
SGPLGSLIRKHL SEQ ID NO: 60
SLIRKHLSGPLG SEQ ID NO: 61
SGPLGSLIRKHLL SEQ ID NO: 62
and
SLIRKHLLSGPLG SEQ ID NO: 63
XXXXXXLFSLIRKHLL (SEQ ID NO: 64)
XXXXXXXLFSLIRKHLL (SEQ ID NO: 65)
XXXXXXXXLFSLIRKHLL (SEQ ID NO: 66)
XXXXXXXXXLFSLIRKHLL (SEQ ID NO: 67)
LFSLIRKHLLXXXXXX (SEQ ID NO: 68)
LFSLIRKHLLXXXXXXX (SEQ ID NO: 69)
LFSLIRKHLLXXXXXXXX (SEQ ID NO: 70)
LFSLIRKHLLXXXXXXXXX (SEQ ID NO: 71)
SGPLGLFSLIRKHLL (SEQ ID NO: 72)
LFSLIRKHLLSGPLG (SEQ ID NO: 73)
wherein X when present is defined as G, S, P, D, N, R,
A Q, C, H, Y, W, T, F, or K foreach position.
KKRRKKRRKKRRKKRRKKRRKKRR SEQ ID NO: 74
RKKRRQ SEQ ID NO: 75

1. A steroid hormone receptor ligand binding peptide and derivatives thereof, comprising at least the contiguous amino acid residues LFSLIR, corresponding to amino acid residues 41 to 46 of SEQ ID NO:3 or amino acid residues 5-10 of SEQ ID NO: 9.

2. The steroid receptor ligand binding peptide according to claim 1 wherein the peptide comprises: 10 contiguous amino acid residues of the carboxyl terminus of SEQ ID NO:3; or the amino acid sequence of SEQ ID NO: 9.

3. (canceled)

4. The steroid hormone receptor ligand binding peptide according to claim 1, wherein the peptide further comprises a cell penetration peptide linked to either the amino or carboxyl terminus.

5. The steroid hormone receptor ligand binding peptide according to claim 4, wherein the cell penetration peptide comprises the amino acid sequence of SEQ ID NO:7, 74, or 75.

6. The steroid hormone receptor ligand binding peptide according to claim 1, wherein the peptide further comprises a disordered loop or ring of 5, 6, 7, 8, 9, or more amino acid residues linked to either the amino or carboxyl terminus of the peptide, wherein the amino acid residues form a flexible non-helical, cell permeable, and ring forming sequence, wherein the amino acid residues comprise G, S, P, D, N, R, A, Q, C, H, Y, W, T, F, or K, and wherein the peptide is selected from:
(SEQ ID NO: 40)
X*XXXXSLIRKHL*,
(SEQ ID NO: 41)
X*XXXXSLIRKHLL*,
(SEQ ID NO: 42)
SLIRKHLXXXXX*,
(SEQ ID NO: 43)
SLIRKHLLXXXXX*,
(SEQ ID NO: 44)
X*XXXXXSLIRKHL*,
(SEQ ID NO: 45)
X*XXXXXSLIRKHLL*,
(SEQ ID NO: 46)
SLIRKHLXXXXXX*,
(SEQ ID NO: 47)
SLIRKHLLXXXXXX*,
(SEQ ID NO: 48)
XXXXXXXXSLIRKHL*,
(SEQ ID NO: 49)
XXXXXXXXSLIRKHLL*,
(SEQ ID NO: 50)
SLIRKHLXXXXXXX*,
(SEQ ID NO: 51)
SLIRKHLLXXXXXXX,
(SEQ ID NO: 52)
X*XXXXXXXSLIRKHL*,
(SEQ ID NO: 53)
X*XXXXXXXSLIRKHLL*,
(SEQ ID NO: 54)
S*LIRKHLXXXXXXXX*,
(SEQ ID NO: 55)
S*LIRKHLLXXXXXXXX*
(SEQ ID NO: 56)
X*XXXXXXXXSLIRKHL*
(SEQ ID NO: 57)
X*XXXXXXXXSLIRKHLL*,
(SEQ ID NO: 58)
S*LIRKHLXXXXXXXXX*,
(SEQ ID NO: 59)
S*LIRKHLLXXXXXXXXX*,
(SEQ ID NO: 64)
X*XXXXXLFSLIRKHLL*,
(SEQ ID NO: 65)
X*XXXXXXLFSLIRKHLL*,
(SEQ ID NO: 66)
X*XXXXXXXLFSLIRKHLL*,
(SEQ ID NO: 67)
X*XXXXXXXXLFSLIRKHLL*,
(SEQ ID NO: 68)
L*FSLIRKHLLXXXXXX*,
(SEQ ID NO: 69)
L*FSLIRKHLLXXXXXXX*,
(SEQ ID NO: 70)
L*FSLIRKHLLXXXXXXXX*,
and
(SEQ ID NO: 71)
L*FSLIRKHLLXXXXXXXXX*.
wherein X designates any amino acid, and * designates an optional amide bond to cyclize the peptide, and wherein the peptide is cyclized.

7. The steroid hormone receptor ligand binding peptide according to claim 6, wherein the peptide is selected from:
(SEQ ID NO: 60)
S*GPLGSLIRKHL*,
(SEQ ID NO: 61)
S*LIRKHLSGPLG*,
(SEQ ID NO: 62)
S*GPLGSLIRKHLL*,
(SEQ ID NO: 63)
S*LIRKHLLSGPLG*,
(SEQ ID NO: 72)
S*GPLGLFSLIRKHLL*,
and
(SEQ ID NO: 73)
L*FSLIRKHLLSGPLG*

8. (canceled)

9. The steroid hormone receptor ligand binding peptide according to claim 1, wherein the peptide comprises a stapled peptide comprising the amino acid sequence: PPX³VLFSLIX¹⁰KHLLRKKRRQRRRG (SEQ ID NO:38) or PPX³VLFSLIX¹⁰KHHL (SEQ ID NO:39), wherein X³ is R-2-(7′-octenyl) alanine and X¹⁰ is S-2-(4′-pentenyl) alanine which form an intrachain bond.

10. The steroid hormone receptor ligand binding peptide according to claim 1, wherein the steroid hormone receptor is a human androgen receptor and/or a human estrogen receptor.

11. (canceled)

12. An isolated PRAC1 peptide comprising the amino acid sequence at least about 95%, 90%, 80%, 70%, 60%, 50% identical to the amino acid sequence set forth in any one of SEQ ID NOS: 3, 9, 38, and 39-73.

13. A pharmaceutical composition comprising the peptide of claim 12 and a pharmaceutically acceptable carrier.

14. A polynucleotide encoding the peptide of claim 12.

15. A vector comprising the polynucleotide of claim 14.

16. The vector of claim 15, wherein the polynucleotide is operably linked to an expression control sequence, wherein optionally:
(i) the expression vector is capable of delivering the polynucleotide to a cancer cell; and/or
(ii) the expression vector is a viral vector, wherein further optionally, the viral vector is a lentiviral vector or a retroviral vector,
wherein the cancer cell is a nuclear hormone receptor superfamily driven cancer.

17-18. (canceled)

19. A method of treating a steroid hormone driven cancer in a subject, the method comprising administering to the subject a therapeutically effective amount of the pharmaceutical composition of claim 13, wherein the steroid hormone driven cancer is an androgen receptor positive prostate cancer or an estrogen receptor positive uterine, ovarian, or breast cancer.

20. The method of claim 19, wherein the prostate cancer is a metastatic castration resistant prostate cancer, and the uterine, ovarian, or breast cancer are a metastatic uterine, ovarian, or breast cancer.

21-23. (canceled)

24. The method according to claim 19, wherein the pharmaceutical composition is administered in combination with another therapy.

25. The method according to claim 24, wherein:
(i) the cancer is prostate cancer and the other therapy comprises a therapy targeting an androgen receptor signaling axis, a therapy targeting poly(ADP-ribose)phosphatase, a therapy directed against a cell surface target, a therapy using a radioligand, a therapy comprising a chemotherapeutic drug, a therapy comprising the use of external beam radiation, brachytherapy, or surgery, or an immunotherapy; or
(ii) the cancer is uterine cancer, ovarian cancer, or breast cancer and the other therapy comprises a hormonal therapy, a chemotherapy, a targeted therapy, a therapy directed against a cell surface target, a therapy using external beam radiation, brachytherapy or surgery, or an immunotherapy.

26-28. (canceled)

29. A method for inhibiting the interaction of a steroid hormone receptor, one or more of its co-factors, and its ligand to reduce steroid hormone-driven gene activation, comprising contacting the steroid hormone receptor with an effective amount of a peptide according to claim 12, wherein the steroid hormone receptor is an androgen receptor, or an estrogen receptor.

30-33. (canceled)

34. A method for predicting response to a therapy targeting an androgen receptor signaling axis for a subject suffering from a steroid hormone driven cancer, the method comprising:
(i) obtaining one or more biological samples from the subject;
(ii) measuring the expression level of at least one biomarker gene in the one or more biological samples;
(iii) comparing the expression levels of the at least one biomarker in the one or more biological sample obtained from the subject with an expression level of the at least one biomarker in a reference/control sample; and
(iv) identifying the subject as responsive or non-responsive to the therapy targeting the androgen signaling pathway when the at least one biomarker gene is differentially expressed in the biological sample obtained from the subject relative to the expression levels of the biomarker in the reference or control sample,
wherein the at least one biomarker gene comprises Prostate Cancer Susceptibility Candidate 1 (PRAC1).

35. The method of claim 34, wherein the reference/control sample is a corresponding biological sample obtained from a healthy subject not suffering from prostate cancer, or from a subject not suffering from androgen resistant prostate cancer.

36-37. (canceled)

38. A method of overcoming resistance or restoring sensitivity to a therapy targeting an androgen receptor signaling pathway in a subject suffering from a steroid hormone driven cancer, the method comprising:
administering to the subject an effective amount of the peptide of claim 12, wherein the subject is identified as resistant to the therapy targeting the androgen receptor signaling pathway by a method comprising:
(i) obtaining one or more biological samples from the subject;
(ii) measuring the expression level of at least one biomarker gene comprising PRAC1 in the one or more biological samples;
(iii) comparing the expression levels of the at least one biomarker gene comprising PRAC1 in the one or more biological sample obtained from the subject with an expression level of PRAC1 in a reference/control sample;
wherein a lower expression of PRAC1 (PRAC1^{low}) in the one or more biological sample obtained from the subject relative to the reference/control sample identifies the subject as resistant to the therapy targeting the androgen receptor signaling pathway, and wherein the reference/control sample is a corresponding biological sample obtained from a healthy subject or a subject not suffering from androgen resistant prostate cancer.


SEQUENCE LISTING (75 sequences)

SEQ ID NO: 1 - DNA, 403 bp, Homo sapiens
  source 1..403: mol_type = other DNA
gtccttcctctcctagcctaaggcgtgcaaacagagcgccactgggaggctgaaacctttaggccgatgcttgcttgcaaggtcaggcaagctggattctggtccccacctttgcagagagaacagcgatgttgtgcgcccatttctcagatcaaggaccggcccatcttactacctccaagagtgcttttctctctaataagaaaacatctactttgaaacatctactgggcgagaccaggagtgatggctcagcctgtaattctggaatttcgggaggccgaggcaggaagattccttgagcacaggagttccagaccagcctgggcaatgtagcaagacgctgtctctatttatacaataaaatttttttaaaaaaggaaaaaaaaaaaaaaaaaaaaaa

SEQ ID NO: 2 - DNA, 153 bp, Homo sapiens
  source 1..153: mol_type = other DNA
atgcttgcttgcaaggtcaggcaagctggattctggtccccacctttgcagagagaacagcgatgttgtgcgcccatttctcagatcaaggaccggcccatcttactacctccaagagtgcttttctctctaataagaaaacatctactttga

SEQ ID NO: 3 - protein, 50 aa, Homo sapiens
MLACKVRQAGFWSPPLQREQRCCAPISQIKDRPILLPPRVLFSLIRKHLL

SEQ ID NO: 4 - DNA, 174 bp, Homo sapiens
  source 1..174: mol_type = other DNA
atgttgtgcgcccatttctcagatcaaggaccggcccatcttactacctccaagagtgcttttctctctaataagaaaacatctactttgaaacatctactgggcgagaccaggagtgatggctcagcctgtaattctggaatttcgggaggccgaggcaggaagattccttga

SEQ ID NO: 5 - protein, 57 aa, Homo sapiens
MLCAHFSDQGPAHLTTSKSAFLSNKKTSTLKHLLGETRSDGSACNSGISGGRGRKIP

SEQ ID NO: 6 - DNA, 153 bp, synthetic construct
  source 1..153: mol_type = other DNA
atgttggcgtgtaaagtgcgccaggcgggcttttggagtcccccgctccaacgcgagcaaaggtgctgcgccccgataagccaaataaaagatagaccgatactcttgccgccccgcgtcttgtttagcttgatccgcaagcacttgttgtga

SEQ ID NO: 7 - protein, 9 aa, synthetic construct
RKKRRQRRG

SEQ ID NO: 8 - protein, 24 aa, synthetic construct
PPRVLFSLIRKHLLRKKRRQRRRG

SEQ ID NO: 9 - protein, 14 aa, synthetic construct
PPRVLFSLIRKHLL

SEQ ID NO: 10 - protein, 16 aa, synthetic construct
LFSLIRKHLLRKKRRQ

SEQ ID NO: 11 - protein, 60 aa, synthetic construct
MLACKVRQAGFWSPPLQREQRCCAPISQIKDRPILLPPRVLFSLIRKHLLRKKRRQRRRG

SEQ ID NO: 12 - protein, 20 aa, synthetic construct
MLACKVRQAGRKKRRQRRRG

SEQ ID NO: 13 - protein, 20 aa, synthetic construct
AGFWSPPLQRRKKRRQRRRG

SEQ ID NO: 14 - protein, 20 aa, synthetic construct
QREQRCCAPIRKKRRQRRRG

SEQ ID NO: 15 - protein, 20 aa, synthetic construct
PISQIKDRPIRKKRRQRRRG

SEQ ID NO: 16 - protein, 20 aa, synthetic construct
PILLPPRVLFRKKRRQRRRG

SEQ ID NO: 17 - protein, 20 aa, synthetic construct
LFSLIRKHLLRKKRRQRRRG

SEQ ID NO: 18 - protein, 24 aa, synthetic construct
PPRVLFSLIRKHLLRKKRRQRRRG

SEQ ID NO: 19 - protein, 24 aa, synthetic construct
APRVLFSLIRKHLLRKKRRQRRRG

SEQ ID NO: 20 - protein, 24 aa, synthetic construct
PARVLFSLIRKHLLRKKRRQRRRG

SEQ ID NO: 21 - protein, 24 aa, synthetic construct
PPAVLFSLIRKHLLRKKRRQRRRG

SEQ ID NO: 22 - protein, 24 aa, synthetic construct
PPRALFSLIRKHLLRKKRRQRRRG

SEQ ID NO: 23 - protein, 24 aa, synthetic construct
PPRVAFSLIRKHLLRKKRRQRRRG

SEQ ID NO: 24 - protein, 24 aa, synthetic construct
PPRVLASLIRKHLLRKKRRQRRRG

SEQ ID NO: 25 - protein, 24 aa, synthetic construct
PPRVLFALIRKHLLRKKRRQRRRG

SEQ ID NO: 26 - protein, 24 aa, synthetic construct
PPRVLFSAIRKHLLRKKRRQRRRG

SEQ ID NO: 27 - protein, 24 aa, synthetic construct
PPRVLFSLARKHLLRKKRRQRRRG

SEQ ID NO: 28 - protein, 24 aa, synthetic construct
PPRVLFSLIAKHLLRKKRRQRRRG

SEQ ID NO: 29 - protein, 24 aa, synthetic construct
PPRVLFSLIRAHLLRKKRRQRRRG

SEQ ID NO: 30 - protein, 24 aa, synthetic construct
PPRVLFSLIRKALLRKKRRQRRRG

SEQ ID NO: 31 - protein, 24 aa, synthetic construct
PPRVLFSLIRKHALRKKRRQRRRG

SEQ ID NO: 32 - protein, 24 aa, synthetic construct
PPRVLFSLIRKHLARKKRRQRRRG

SEQ ID NO: 33 - protein, 17 aa, synthetic construct
PPRVLFSLIRKHLLRKK

SEQ ID NO: 34 - protein, 20 aa, synthetic construct
PPRVLFSLIRKHLLRKKRRQ

SEQ ID NO: 35 - protein, 13 aa, synthetic construct
FSLIRKHLLRKKR

SEQ ID NO: 36 - protein, 15 aa, synthetic construct
FSLIRKHLLRKKRRQ

SEQ ID NO: 37 - protein, 10 aa, synthetic construct
LFSLIRKHLL

SEQ ID NO: 38 - protein, 24 aa, synthetic construct
  VARIANT 3: note = R-2-(7 prime-octenyl) alanine
  VARIANT 10: note = S-2-(4 prime-pentenyl) alanine
  SITE 3: note = bond of side chain with amino acid variant at position 10
  SITE 10: note = bond of side chain with amino acid variant at position 3
PPXVLFSLIXKHLLRKKRRQRRRG

SEQ ID NO: 39 - protein, 14 aa, synthetic construct
  VARIANT 3: note = R-2-(7 prime-octenyl) alanine
  VARIANT 10: note = S-2-(4 prime-pentenyl) alanine
  SITE 3: note = bond of side chain with amino acid variant at position 10
  SITE 10: note = bond of side chain with amino acid variant at position 3
PPXVLFSLIXKHLL

SEQ ID NO: 40 - protein, 12 aa, synthetic construct
  VARIANT 1..5: note = X is defined as G, S, P, D, N, R, A, Q, C, H, Y, W, T, F, or K
  SITE 1: note = amide bond with C-terminal amino acid
  SITE 12: note = amide bond with N-terminal amino acid
XXXXXSLIRKHL

SEQ ID NO: 41 - protein, 13 aa, synthetic construct
  VARIANT 1..5: note = X is defined as G, S, P, D, N, R, A, Q, C, H, Y, W, T, F, or K
  SITE 1: note = amide bond with C-terminal amino acid
  SITE 13: note = amide bond with N-terminal amino acid
XXXXXSLIRKHLL

SEQ ID NO: 42 - protein, 12 aa, synthetic construct
  VARIANT 8..12: note = X is defined as G, S, P, D, N, R, A, Q, C, H, Y, W, T, F, or K
  SITE 1: note = amide bond with C-terminal amino acid
  SITE 12: note = amide bond with N-terminal amino acid
SLIRKHLXXXXX

SEQ ID NO: 43 - protein, 13 aa, synthetic construct
  VARIANT 9..13: note = X is defined as G, S, P, D, N, R, A, Q, C, H, Y, W, T, F, or K
  SITE 1: note = amide bond with C-terminal amino acid
  SITE 13: note = amide bond with N-terminal amino acid
SLIRKHLLXXXXX

SEQ ID NO: 44 - protein, 13 aa, synthetic construct
  VARIANT 1..6: note = X is defined as G, S, P, D, N, R, A, Q, C, H, Y, W, T, F, or K
  SITE 1: note = amide bond with C-terminal amino acid
  SITE 13: note = amide bond with N-terminal amino acid
XXXXXXSLIRKHL

SEQ ID NO: 45 - protein, 14 aa, synthetic construct
  VARIANT 1..6: note = X is defined as G, S, P, D, N, R, A, Q, C, H, Y, W, T, F, or K
  SITE 1: note = amide bond with C-terminal amino acid
  SITE 14: note = amide bond with N-terminal amino acid
XXXXXXSLIRKHLL

SEQ ID NO: 46 - protein, 13 aa, synthetic construct
  VARIANT 8..13: note = X is defined as G, S, P, D, N, R, A, Q, C, H, Y, W, T, F, or K
  SITE 1: note = amide bond with C-terminal amino acid
  SITE 13: note = amide bond with N-terminal amino acid
SLIRKHLXXXXXX

SEQ ID NO: 47 - protein, 14 aa, synthetic construct
  VARIANT 9..14: note = X is defined as G, S, P, D, N, R, A, Q, C, H, Y, W, T, F, or K
  SITE 1: note = amide bond with C-terminal amino acid
  SITE 14: note = amide bond with N-terminal amino acid
SLIRKHLLXXXXXX

SEQ ID NO: 48 - protein, 14 aa, synthetic construct
  VARIANT 1..7: note = X is defined as G, S, P, D, N, R, A, Q, C, H, Y, W, T, F, or K
  SITE 1: note = amide bond with C-terminal amino acid
  SITE 14: note = amide bond with N-terminal amino acid
XXXXXXXSLIRKHL

SEQ ID NO: 49 - protein, 15 aa, synthetic construct
  VARIANT 1..7: note = X is defined as G, S, P, D, N, R, A, Q, C, H, Y, W, T, F, or K
  SITE 1: note = amide bond with C-terminal amino acid
  SITE 15: note = amide bond with N-terminal amino acid
XXXXXXXSLIRKHLL

SEQ ID NO: 50 - protein, 14 aa, synthetic construct
  VARIANT 8..14: note = X is defined as G, S, P, D, N, R, A, Q, C, H, Y, W, T, F, or K
  SITE 1: note = amide bond with C-terminal amino acid
  SITE 14: note = amide bond with N-terminal amino acid
SLIRKHLXXXXXXX

SEQ ID NO: 51 - protein, 15 aa, synthetic construct
  VARIANT 9..15: note = X is defined as G, S, P, D, N, R, A, Q, C, H, Y, W, T, F, or K
  SITE 1: note = amide bond with C-terminal amino acid
  SITE 15: note = amide bond with N-terminal amino acid
SLIRKHLLXXXXXXX

SEQ ID NO: 52 - protein, 15 aa, synthetic construct
  VARIANT 1..8: note = X is defined as G, S, P, D, N, R, A, Q, C, H, Y, W, T, F, or K
  SITE 1: note = amide bond with C-terminal amino acid
  SITE 15: note = amide bond with N-terminal amino acid
XXXXXXXXSLIRKHL

SEQ ID NO: 53 - protein, 16 aa, synthetic construct
  VARIANT 1..8: note = X is defined as G, S, P, D, N, R, A, Q, C, H, Y, W, T, F, or K
  SITE 1: note = amide bond with C-terminal amino acid
  SITE 16: note = amide bond with N-terminal amino acid
XXXXXXXXSLIRKHLL

SEQ ID NO: 54 - protein, 15 aa, synthetic construct
  VARIANT 8..15: note = X is defined as G, S, P, D, N, R, A, Q, C, H, Y, W, T, F, or K
  SITE 1: note = amide bond with C-terminal amino acid
  SITE 15: note = amide bond with N-terminal amino acid
SLIRKHLXXXXXXXX

SEQ ID NO: 55 - protein, 16 aa, synthetic construct
  VARIANT 9..16: note = X is defined as G, S, P, D, N, R, A, Q, C, H, Y, W, T, F, or K
  SITE 1: note = amide bond with C-terminal amino acid
  SITE 16: note = amide bond with N-terminal amino acid
SLIRKHLLXXXXXXXX

SEQ ID NO: 56 - protein, 16 aa, synthetic construct
  VARIANT 1..9: note = X is defined as G, S, P, D, N, R, A, Q, C, H, Y, W, T, F, or K
  SITE 1: note = amide bond with C-terminal amino acid
  SITE 16: note = amide bond with N-terminal amino acid
XXXXXXXXXSLIRKHL

SEQ ID NO: 57 - protein, 17 aa, synthetic construct
  VARIANT 1..9: note = X is defined as G, S, P, D, N, R, A, Q, C, H, Y, W, T, F, or K
  SITE 1: note = amide bond with C-terminal amino acid
  SITE 17: note = amide bond with N-terminal amino acid
XXXXXXXXXSLIRKHLL

SEQ ID NO: 58 - protein, 16 aa, synthetic construct
  VARIANT 8..16: note = X is defined as G, S, P, D, N, R, A, Q, C, H, Y, W, T, F, or K
  SITE 1: note = amide bond with C-terminal amino acid
  SITE 16: note = amide bond with N-terminal amino acid
SLIRKHLXXXXXXXXX

SEQ ID NO: 59 - protein, 17 aa, synthetic construct
  VARIANT 9..17: note = X is defined as G, S, P, D, N, R, A, Q, C, H, Y, W, T, F, or K
  SITE 1: note = amide bond with C-terminal amino acid
  SITE 17: note = amide bond with N-terminal amino acid
SLIRKHLLXXXXXXXXX

SEQ ID NO: 60 - protein, 12 aa, synthetic construct
  SITE 1: note = amide bond with C-terminal amino acid
  SITE 12: note = amide bond with N-terminal amino acid
SGPLGSLIRKHL

SEQ ID NO: 61 - protein, 12 aa, synthetic construct
  SITE 1: note = amide bond with C-terminal amino acid
  SITE 12: note = amide bond with N-terminal amino acid
SLIRKHLSGPLG

SEQ ID NO: 62 - protein, 13 aa, synthetic construct
  SITE 1: note = amide bond with C-terminal amino acid
  SITE 13: note = amide bond with N-terminal amino acid
SGPLGSLIRKHLL

SEQ ID NO: 63 - protein, 13 aa, synthetic construct
  SITE 1: note = amide bond with C-terminal amino acid
  SITE 13: note = amide bond with N-terminal amino acid
SLIRKHLLSGPLG

SEQ ID NO: 64 - protein, 16 aa, synthetic construct
  VARIANT 1..6: note = X is defined as G, S, P, D, N, R, A, Q, C, H, Y, W, T, F, or K
  SITE 1: note = amide bond with C-terminal amino acid
  SITE 16: note = amide bond with N-terminal amino acid
XXXXXXLFSLIRKHLL

SEQ ID NO: 65 - protein, 17 aa, synthetic construct
  VARIANT 1..7: note = X is defined as G, S, P, D, N, R, A, Q, C, H, Y, W, T, F, or K
  SITE 1: note = amide bond with C-terminal amino acid
  SITE 17: note = amide bond with N-terminal amino acid
XXXXXXXLFSLIRKHLL

SEQ ID NO: 66 - protein, 18 aa, synthetic construct
  VARIANT 1..8: note = X is defined as G, S, P, D, N, R, A, Q, C, H, Y, W, T, F, or K
  SITE 1: note = amide bond with C-terminal amino acid
  SITE 18: note = amide bond with N-terminal amino acid
XXXXXXXXLFSLIRKHLL

SEQ ID NO: 67 - protein, 19 aa, synthetic construct
  VARIANT 1..9: note = X is defined as G, S, P, D, N, R, A, Q, C, H, Y, W, T, F, or K
  SITE 1: note = amide bond with C-terminal amino acid
  SITE 19: note = amide bond with N-terminal amino acid
XXXXXXXXXLFSLIRKHLL

SEQ ID NO: 68 - protein, 16 aa, synthetic construct
  VARIANT 11..16: note = X is defined as G, S, P, D, N, R, A, Q, C, H, Y, W, T, F, or K
  SITE 1: note = amide bond with C-terminal amino acid
  SITE 16: note = amide bond with N-terminal amino acid
LFSLIRKHLLXXXXXX

SEQ ID NO: 69 - protein, 17 aa, synthetic construct
  VARIANT 11..17: note = X is defined as G, S, P, D, N, R, A, Q, C, H, Y, W, T, F, or K
  SITE 1: note = amide bond with C-terminal amino acid
  SITE 17: note = amide bond with N-terminal amino acid
LFSLIRKHLLXXXXXXX

SEQ ID NO: 70 - protein, 18 aa, synthetic construct
  VARIANT 11..18: note = X is defined as G, S, P, D, N, R, A, Q, C, H, Y, W, T, F, or K
  SITE 1: note = amide bond with C-terminal amino acid
  SITE 18: note = amide bond with N-terminal amino acid
LFSLIRKHLLXXXXXXXX

SEQ ID NO: 71 - protein, 19 aa, synthetic construct
  VARIANT 11..19: note = X is defined as G, S, P, D, N, R, A, Q, C, H, Y, W, T, F, or K
  SITE 1: note = amide bond with C-terminal amino acid
  SITE 19: note = amide bond with N-terminal amino acid
LFSLIRKHLLXXXXXXXXX

SEQ ID NO: 72 - protein, 15 aa, synthetic construct
  SITE 1: note = amide bond with C-terminal amino acid
  SITE 15: note = amide bond with N-terminal amino acid
SGPLGLFSLIRKHLL

SEQ ID NO: 73 - protein, 15 aa, synthetic construct
  SITE 1: note = amide bond with C-terminal amino acid
  SITE 15: note = amide bond with N-terminal amino acid
LFSLIRKHLLSGPLG

SEQ ID NO: 74 - protein, 24 aa, synthetic construct
  SITE 1: note = amide bond with C-terminal amino acid
  SITE 24: note = amide bond with N-terminal amino acid
KKRRKKRRKKRRKKRRKKRRKKRR

SEQ ID NO: 75 - protein, 6 aa, synthetic construct
  SITE 1: note = amide bond with C-terminal amino acid
  SITE 6: note = amide bond with N-terminal amino acid
RKKRRQ